(19) United States
(12) Patent Application Publication
(10) Pub. No.: US 2018/0353770 A1
(43) Pub. Date: Dec. 13, 2018

(54) SYSTEMS AND METHODS FOR TARGETED UVB PHOTOTHERAPY FOR AUTOIMMUNE DISORDERS AND OTHER INDICATIONS

(71) Applicant: BENESOL, INC., Bainbridge Island, WA (US)

(72) Inventor: William A. Moffat, Bainbridge Island, WA (US)

(21) Appl. No.: 15/569,019

(22) PCT Filed: Apr. 27, 2016
(86) PCT No.: PCT/US16/29615

§ 371 Date: Oct. 24, 2017

Related U.S. Application Data
(60) Provisional application No. 62/153,426, filed on Apr. 27, 2015.
(60) Provisional application No. 62/198,084, filed on Jul. 28, 2015.

(51) Int. Cl. A61N 5/06

(52) U.S. Cl. CPC A61N 5/0616; A61N 2005/0667; A61N 2005/0661; A61N 2005/0654; A61N 2005/0645; A61N 2005/0626; A61N 2005/0652

(57) ABSTRACT

The present disclosure is directed to systems and methods for targeted UVB phototherapy for treating autoimmune disorders and other indications. In one embodiment, a phototherapeutic system can include a radiation source configured to emit light. At least 75% of the light emitted by the radiation source can have a target wavelength range with a bandwidth between 298 nm and 307 nm. The phototherapeutic system can also include a controller operably connected to the radiation source and configured to determine a dosage for a phototherapy session. Dosage can correspond to a product of the intensity of the radiation source and an exposure time of the radiation source, and may have an upper bound less than 1 minimal erythema dose (MED). Delivery of the dose of phototherapy can stimulate an immune response to treat an autoimmune disorder.

### CROSS-REFERENCE TO RELATED APPLICATIONS

The present application claims priority to U.S. Provisional Patent Application No. 62/153,426, filed Apr. 27, 2015, and U.S. Provisional Patent Application No. 62/198,084, filed Jul. 28, 2015, both of which are incorporated by reference herein in their entireties.

### TECHNICAL FIELD

The present technology relates to phototherapy, and more particularly to UVB phototherapy.

### BACKGROUND

Autoimmune and immune-mediated diseases are defined by an abnormal immune response of the body against substances and tissues normally present in the body, resulting in the destruction of health body tissue. Thus, an autoimmune disorder occurs when the body's immune system attacks and destroys healthy body tissue by mistake. There are more than 80 types of autoimmune disorders, some of the more common including Multiple Sclerosis (“MS”), Rheumatoid Arthritis (“RA”), Type 1 Diabetes mellitus (“T1D”), Ulcerative Colitis (“UC”), Crohn's Disease (“CD”), Celiac and Lupus. The exact cause of autoimmune disorders is still not fully known, but many are thought to have both genetic and environmental components.

MS is a chronic autoimmune disease characterized by inflammation, demyelination, and axonal degeneration of central nervous system which disrupts the flow of information within the brain, and between the brain and body. There is no cure for this debilitating disease, and the cause is linked to genetic susceptibility and environmental factors, including UVB sun exposure and vitamin D. Symptoms of MS usually appear in episodic acute relapse periods (known as “attacks” or “flares”), with breaks of remission, in a gradual progressive deterioration of neurological function. Although fatigue and pain are two of the most common symptoms in MS patients, there is a wide range of symptoms, including weakness, numbness, dizziness, depression, cognition, and problems with bowel, bladder, vision, and walking.

RA is a chronic, systemic inflammatory disorder of the joints that may affect surrounding tissues and organs. Although the cause of this autoimmune disease is still not fully understood, there is evidence linking genetics in combination with environmental factors such as infection, sun exposure, and hormonal changes. The primary symptoms are joints that are painful, stiff, and have loss in range of motion. Other symptoms can include sleep difficulties, chest pain, dry eyes and mouth, itchy or burning eyes, and tingling or burning in the hands or feet.

Celiac disease is an autoimmune disorder of the small intestine that occurs in genetically predisposed people of all ages from middle infancy onward. Studies using blood samples indicate that approximately one percent of the population has celiac disease. Symptoms may include chronic diarrhea, failure to thrive (in children), and fatigue. Some people appear to be asymptomatic, yet changes in the bowel make it less able to absorb nutrients, minerals and the fat-soluble vitamins A, D, E. and K. It is well established that dietary vitamin D malabsorption caused by celiac disease frequently leads to vitamin D deficiency and reduced bone mineral density. Studies have shown that celiac disease and resultant vitamin D deficiency can cause osteomalacia or osteoporosis.

CD is a type of inflammatory bowel disease that may affect any part of the gastrointestinal tract from mouth to anus, causing a wide variety of symptoms. It primarily causes abdominal pain, diarrhea, vomiting, weight loss, skin rashes, arthritis, inflammation of the eye, tiredness, and lack of concentration. CD is thought to be the result of a malfunction of the innate immune system, leading to an uncontrolled inflammation of the GI tract caused by a combination of environmental factors and genetic predisposition. The disease commonly results in malnutrition due to carbohydrate and fat malabsorption. Because vitamin D is fat soluble, vitamin D deficiency is common in patients with CD.

T1D is an inflammatory autoimmune disease that causes the destruction of insulin-producing beta cells of the pancreas subsequently leading to increased blood and urine glucose. T1D strikes both children and adults at any age. It comes on suddenly, causes dependence on injected or pumped insulin for life, and carries the constant threat of devastating complications. The classical symptoms are frequent urination, increased thirst, increased hunger, and weight loss.

UC is an inflammatory bowel disease affects the innermost lining of your large intestine that causes long-lasting inflammation and ulcers in the digestive tract. UC is an immune-mediated disease that is caused by a combination of genetic pre-disposition and environmental interaction. Vitamin D malabsorption is common in patients with UC making vitamin D deficiency highly prevalent.

Lupus is a category for a collection of autoimmune diseases in which the body's immune system becomes hyperactive and starts to attack healthy tissues, resulting in inflammation and tissue damage. Four main types of lupus exist: systemic lupus erythematosus, discoid lupus erythematosus, drug-induced lupus erythematosus, and neonatal lupus erythematosus. Of these, systemic lupus erythematosus (“SLE”) is the most common and serious form. The disease can affect almost any part of the body and is characterized by remission and relapses. There is a high prevalence of vitamin D insufficiency/deficiency found in patients with lupus.

Most autoimmune diseases are chronic, but many can be controlled with treatment. Autoimmune diseases are typically treated with immunosuppressive medication that decreases an overactive immune response. Low vitamin D has been identified as a risk factor for the development and severity of several autoimmune diseases. Elevating serum vitamin D concentration is often recommended for patients, but attempting to do so through oral vitamin D supplementation has risks and results of such therapy are inconclusive.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology.

FIG. 1 is a graph illustrating phototherapy emission spectra for various types of UV emitting devices.

FIG. 2 is a graph illustrating the contact hypersensitivity action spectrum.

FIG. 3 is a graph illustrating the cis-urocanic acid action spectrum.

FIG. 4 is a graph illustrating in vivo thymine dimer action spectra.

FIG. 5 is a graph illustrating the in vivo tumor necrosis factor alpha action spectrum.

FIG. 6 is a graph illustrating the immune response action spectra.

FIG. 7 is a graph illustrating an immune response phototherapy action spectrum configured in accordance with embodiments of the present technology.

FIG. 8 is a graph illustrating the pre-vitamin D3 action spectrum.

FIG. 9 is a graph illustrating the pre-vitamin D3 action spectrum and the vitamin D3 action spectrum.

FIG. 10 is a graph illustrating phototherapy emission spectra for various types of UV emitting devices and the vitamin D3 action spectrum.

FIG. 11 is a graph illustrating a calcitriol action spectrum.

FIG. 12 is a graph illustrating the erythema action spectrum.

FIG. 13 is a graph illustrating UVB phototherapy emission spectra, the immune response phototherapy action spectrum of FIG. 7, and the erythema action spectrum of FIG. 12.

FIG. 14 is a graph illustrating the vitamin D₃/calcitriol action spectrum and the immune response phototherapy action spectrum of FIG. 7.

FIG. 15 is a graph illustrating a combined autoimmune phototherapy action spectrum configured in accordance with embodiments of the present technology.

FIG. 16 is a table illustrating the relationship between skin type, Minimum Erythema Dose (MED), Standard Erythema Dose (SED), and Erythemal Effective Radiant Exposure (EERE).

FIGS. 17-31 are dosage tables illustrating skin type dependent parameters of phototherapy sessions for focused UV phototherapy devices having differing spectral irradiances.

FIG. 32 is an isometric view of a high-energy phototherapeutic apparatus or system for focused UV radiation configured in accordance with an embodiment of the present technology.

FIG. 33 is an isometric view of a low-energy phototherapeutic apparatus or system for focused UV radiation configured in accordance with another embodiment of the present technology.

FIG. 34 is a block diagram illustrating an overview of devices on which some implementations of the present technology may operate.

FIG. 35 is a block diagram illustrating an overview of an environment in which some implementations of the present technology may operate.

### DETAILED DESCRIPTION

The present technology is directed to phototherapy devices, systems, and methods that provide specific wavelength-focused UV and are expected to increase or maximize both immune system impact and calcitriol production, as well as reduce total UV exposure. Such systems and methods can improve the efficacy of combination phototherapy for autoimmune diseases. Although many of the embodiments are described below with respect to systems, devices, and methods for treating autoimmune diseases and promoting vitamin D production in the skin, other applications (e.g., phototherapeutic treatment of other indications) and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to Figures.

### Autoimmune Diseases and Environmental Factors

Increased sunlight exposure has been shown to have a positive impact on several autoimmune diseases. Autoimmune dermatologic disorders, such as psoriasis, have been treated with phototherapy due to the local and systemic immunosuppressive effect of ultraviolet light exposure. Like sunlight, phototherapy using UVB can also produce vitamin D. Human systemic immune suppression and vitamin D production are both highly wavelength dependent, with greatest efficiency of both occurring in a very narrow UVB wavelength range. Developing a phototherapy device that isolates and delivers focused UVB within this peak efficiency wavelength range can simultaneously produce maximum immune response and vitamin D production with the least total radiation. It is expected that a phototherapy device using this targeted UVB range can treat autoimmune diseases that impact various systems of the body.

Genome-wide association studies have defined that there is a genetic component in the susceptibility to several autoimmune diseases (e.g., MS). A person can inherit a predisposition for an autoimmune condition. However, there are environmental factors that are thought to also contribute to the risk and severity of those diseases. Two significant environmental factors are sunlight exposure and vitamin D levels during a person's lifetime, including in utero. One study compared the season of birth to risk of four autoimmune diseases (i.e., rheumatoid arthritis, ulcerative colitis, systemic lupus erythematosus, and multiple sclerosis) to explore the correlation to predicted UVB light exposure and vitamin D status during gestation. This study concluded that the risk of all four autoimmune diseases was inversely correlated with predicted second trimester UVB exposure and third trimester vitamin D status. Another study showed season of birth was associated with celiac. Multiple studies have shown that birth month and latitude are risk factors for MS, indicating that both ultraviolet exposure and resultant vitamin D production are involved. Studies focusing on sun exposure habits have shown increased sun exposure, especially before the age of 15 reduces the risk of developing MS. In patients that have MS, increased sun exposure and vitamin D are correlated to decreased severity, relapse rate and mortality.

### Vitamin D₃

Vitamin D₃ is a fat-soluble secosteroid that can be ingested but is primarily made in the skin when exposed to UVB sunlight. Serum vitamin D level is most often a measurement of 25-hydroxyvitamin D (25-OHD), a prehormone that is produced in the liver by hydroxylation of vitamin D₃. Low serum vitamin D level is associated with increased risk to several autoimmune disorders including MS, RA, CD, UC, T1D and lupus. Genetic research focused on the vitamin D receptor has correlated vitamin D with MS, CD, UC, RA, lupus, celiac, and T1D. Increased maternal vitamin D during gestation has been shown to reduce the risk of the offspring developing MS and T1D. It has also been found that elevated vitamin D during childhood and adolescence is effective at prevention of MS. In patients with MS, higher vitamin D levels are associated with a lower relapse rate, disability, disease progression, depressive symptoms and long term memory. In patients with T1D, higher vitamin D status can reduce insulin requirements. Low vitamin D status is associated with disease activity and severity in patients with RA, UC or lupus.

Because humans receive most of their vitamin D from cutaneous production, serum vitamin D level is primarily a measurement of sun exposure and endogenous vitamin D, not ingested supplements or food sources. This is particularly true of patients with CD, UC or celiac that causes fat malabsorption. Studies with CD patients demonstrate that vitamin D status is linked to sun exposure, and dietary supplementation is inadequate to raise serum concentration. Some intervention studies using vitamin D supplementation (e.g., oral supplements) have shown a positive impact on clinical measures of pathology for MS. However, other studies have concluded that digested vitamin D supplementation as a treatment for MS is inconclusive and perhaps ill-advised considering the risk of overdose. Cutaneous synthesis of vitamin D is the most bioavailable source of vitamin D for those with fat malabsorption caused by inflammatory diseases such as CD, UC and celiac. Given overdose risk and malabsorption conditions, it is expected that cutaneous vitamin D production is most useful form of delivery for the prevention and treatment of autoimmune diseases associated with vitamin D (e.g., rather than digested supplements).

Vitamin D overdose or intoxication is only possible through supplemental form. Endogenous production of vitamin D in the skin is controlled by a regulatory process that has been shown that overdose is impossible or at least highly improbable. The clinical signs of vitamin D intoxication may include symptoms originating in different systems: nausea and vomiting, anorexia, abdominal pain, constipation: polydipsia, polyuria, dehydration, nephrolithiasis, nephrocalcinosis, nephrogenic diabetes insipidus, chronic interstitial nephritis, acute and chronic renal failure; hypotonia, paresthesia, confusion, seizures, apathy, coma; arrhythmia, bradycardia, hypertension, cardiomyopathy; muscle weakness, calcification, osteoporosis; and conjunctival calcification Most symptoms of vitamin D intoxication can be reversed by discontinuing supplementation, however renal damage is only partially reversible. It is common practice to prescribe high-dose cholecalciferol to MS patients but physicians need to be attentive to the possibility of hypercalcemia in patients treated with both high-dose cholecalciferol and calcium. Furthermore, the symptoms of an MS flare are similar to vitamin D overdose which makes proper diagnosis more difficult for practitioners. Gastrointestinal adverse events are the most common side effect for vitamin D supplementation in patients. Cutaneous vitamin D production is most useful form of delivery to optimize serum vitamin D level while preventing adverse events associated with supplementation.

### Calcitriol

The most abundant vitamin D metabolite in the human body is 25-hydroxy vitamin D (25-OHD), but is biologically inert and requires additional hydroxylation to become the active form of vitamin D called calcitriol. This is the biologically active metabolite, with most biological effects mediated through binding to the vitamin D receptor throughout the body including the immune system. Experimental research in vitro and in vivo animal models has further clarified the interaction of calcitriol with the immune system. The evidence obtained from these studies strongly supports a model in which calcitriol mediates a shift to a more anti-inflammatory immune response, and in particular to enhanced regulatory T cell functionality. Studies have found that patients with MS have lower 25-OHD and calcitriol levels than healthy controls. Experimental autoimmune encephalomyelitis (EAE) is an autoimmune disease used by scientists as a standard animal model for the human disease MS. Several studies using the EAE model have demonstrated that calcitriol can prevent development, block progression, and even reverse the disease. Experimental research has shown that calcitriol can prevent or reduce joint destruction in RA. Because exogenous calcitriol administration can rapidly lead to hypercalcemia, endogenous cutaneous production is most useful form of delivery for the prevention and treatment of autoimmune diseases associated with calcitriol.

### Ultraviolet Exposure

Although low vitamin D₃ levels have been associated with increased prevalence and progression of human autoimmune diseases, the benefits of supplementation with vitamin D₃ have not been definitive. Population studies have repeatedly demonstrated that sun exposure is a larger contributor of serum vitamin D concentration than oral consumption. Because humans obtain the vast majority of their vitamin D₃ through exposure of skin to UVB sunlight, vitamin D levels are a measure of past sun exposure more than isolated 25-OHD in a blood test. Sun exposure leads to a systemic immune response that produces several hormones and peptides along with vitamin D. Both vitamin D-dependent and vitamin D-independent pathways have been implicated in the mechanisms of UVB-induced systemic suppression of immunity, which play s a role in controlling autoimmune diseases. In vive human studies have shown that UVB light creates a systemic immune reaction that attenuates systemic autoimmunity via the induction of skin-derived dendritic cells and regulatory T cells. These studies specifically demonstrated the UVB induced mechanism for immune system and anti-inflammatory balance in both autoimmune dermatologic disorders and MS.

There have been several studies that show the positive impact UVB has on autoimmune diseases independent of vitamin D. One study found that the Multiple Sclerosis Severity Score (“MSSS”) had a stronger inverse association with frequent sunlight exposure than vitamin D consumption. Similarly, MRI measures of neurodegeneration in MS are associated with summer sun exposure independent of 25-OHD measurement. One study determined low infant sun exposure was associated with a two-fold increase in T1D. Seasonal variation and duration of sun exposure are both correlated to disease activity in patients with RA. Duration of sun exposure is inversely correlated to the incidence and severity of disease activity in CD. Another study with MS patients showed that higher levels of reported sun exposure, rather than 25-OHD levels, were associated with less depressive symptoms and levels of fatigue. Using the EAE animal model of MS, scientists have found that UVB light can prevent and suppress the disease independent of vitamin D.

### Photoproducts

Substances made from a photochemical reaction are known as photoproducts. When human skin is exposed to sunlight it produces several hormones and peptides. While vitamin D is generally the most recognized health benefit humans receive from sun exposure, it is just one of many important photoproducts that have systemic impact on the human body. The photoproducts Adrenocorticotropic Hormone (ACTH), Melanocyte Stimulating Hormone (MSH) and Beta Endorphin (BE) have a particular positive impact on autoimmune diseases and are all made in the same UVB range as vitamin D3.

Adrenocorticotropic Hormone (“ACTH”) is a peptide hormone secreted by the pituitary gland and by the melanocytes and keratinocytes of the skin when exposed to the UVB spectrum of sunlight. Its principal effects are to increase natural production and release of corticosteroids. It has been established for several decades that ACTH is a powerful anti-inflammatory agent that reduces inflammation throughout the body. Additionally, ACTH acts as an important regulator of the immune system by altering cellular activity of white blood cells, the body's primary defense against both infectious disease and foreign materials. The anti-inflammatory nature of ACTH has made it a preferred treatment option for gout (acute inflammatory arthritis). The combination of anti-inflammatory and immune regulator has made ACTH an established treatment for acute relapses in MS and a target of therapeutic research related to RA.

Melanocyte Stimulating Hormone (MSH) is a peptide hormone secreted by the pituitary gland and by the melanocytes and keratinocytes of the skin when exposed to the UVB spectrum of sunlight. Research has shown that increased MSH reduces appetite and can positively impact metabolism by increasing sensitivity to insulin. MSH is part of the human body immune response to inflammation and infection. This hormone helps regulate the immune system, having properties of an anti-inflammatory, antipyretic and antimicrobial. Several studies have demonstrated MSH exhibiting anti-inflammatory activity in experimental animal models of autoimmune diseases. These studies indicate that MSH can ameliorate disease activity and morbidity in lupus. MS, diabetes, arthritis and UC.

Beta Endorphin (“BE”) is a naturally occurring opioid neuropeptide produced by neurons in the nervous system which binds to the same receptor in the body that is activated by morphine. Naturally produced BE is at least 17 times more potent than morphine, meaning that even small increases in the body can have a profound effect. The production of BE is part of an immune response to inflammation. As such, the endogenous production of BE can be important for inflammation pain management in autoimmune conditions like MS, RA, UC, lupus and Crohn's disease. Some studies have demonstrated that BE has a positive anti-inflammatory immunosuppressive impact on MS and collagen induced arthritis.

BE is produced in the skin by the amino acid precursor pro-opiomelanocortin. UV, not the visual spectrum of sunlight, causes the production and release of BE from the skin. Furthermore, studies have demonstrated that UVB spectrum is far more efficient at producing BE release from the skin than UVA spectrum. Specific studies have shown that blocking BE with a drug used for treatment of opioid dependence even induced withdrawal symptoms in frequent tanners and mice exposed to solar spectrum. Thus, the production of BE from sunlight is expected to be a major contributing factor to less depressive symptoms and fatigue in MS patients.

### Phototherapy Treatment

Autoimmune dermatologic disorders, such as psoriasis and atopic dermatitis (eczema) have been treated with ultraviolet phototherapy. Because many dermatologic disorders are caused by a dysfunction of the dermal immune system, phototherapy efficacy on such conditions is attributed to the local and systemic immune system impact of ultraviolet light. Phototherapy is an efficacious and popular treatment option for all autoimmune related dermatologic disorders.

There are several local and systemic immune-modulating biological mechanisms that contribute to the efficacy of this treatment. Phototherapy has been shown to systemically alter the helper T cell-derived cytokine profile to suppress the dysfunctional overactive immune response. Full body UVA and/or UVB irradiation will cause production of cis-urocanic acid and DNA pyrimidine dimers which leads to systemic immune suppression, considered an effective tool for restoring immune function. Ultraviolet phototherapy has been used to treat various autoimmune dermatologic disorders including psoriasis, atopic dermatitis, vitiligo, chronic urticaria, lichen planus, cutaneous T cell lymphoma, pityriasis lichenoides, parapsoriasis, pityriasis rosea, pruritus, seborrheic dermatitis, actinic prurigo, and alopecia areata. Considering the immune response from UV phototherapy is not just local, but systemic, autoimmune conditions in other systems of the body are expected to respond to the same or similar immune-modulating biological mechanisms that work in the skin. Phototherapy using UVB can produce vitamin D3 and calcitriol as well as initiate an immune response that leads to the production of ACTH, MSH and BE, all of which have been shown to have a positive impact on several non-dermal autoimmune conditions.

### Phototherapy Sources

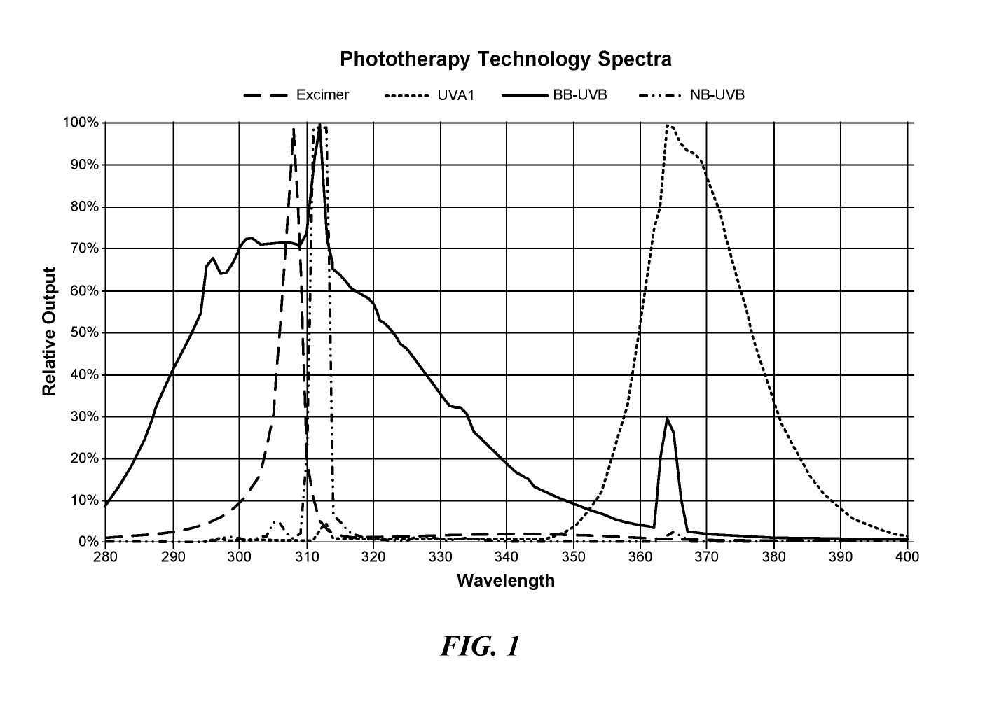

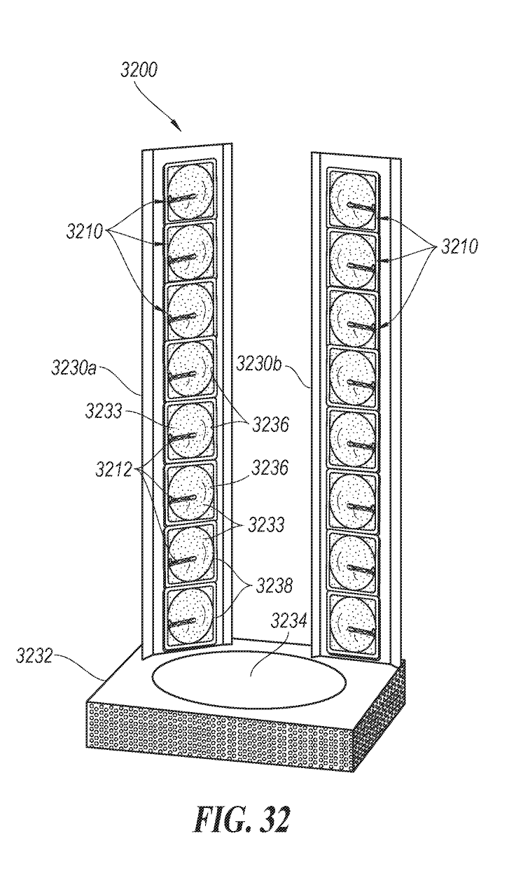

Ultraviolet exposure at various wavelengths has been found to have a beneficial impact on autoimmune dermatological disorders. Many phototherapy devices have been created to provide controlled delivery of ultraviolet radiation using various wavelength combinations, including: Broadband UVB (280-320 nm); narrowband UVB (311-313 nm); excimer laser (308 nm); UVA (340-400 nm); and UVA with psoralen (PUVA). Each technology provides a different spectrum of UV light, but all work on the same principle of immune suppression. It has been demonstrated that systemic immune suppression can be achieved with broadband UVB (BB-UVB), narrow-band UVB (NB-UVB), and PUVA, which uses psoralen as a photosensitizer and subsequent UVA exposure. The spectral analysis of excimer lamps, UVA devices, broad-band UVB devices, and narrow-band UVB devices is shown in FIG. 1. As described in further detail below with reference to FIGS. 32 and 333, these phototherapeutic radiation sources can be incorporated into phototherapeutic devices and systems.

### Immune Response Action Spectra

An action spectrum is the rate of a physiological activity plotted against wavelength of light. It shows which wavelength of light is most effective at producing a photochemical reaction. Action spectra are constructed by measuring a specific biologic response to each wavelength of light using the same amount of radiance density (number of photons). The result is represented using a relative scale, where a wavelength response measurement of 100% represents maximum biological response per photon and 50% at another wavelength would require twice the number of photons to achieve the same biological response. The physiological activities of vitamin D creation, calcitriol synthesis, and systemic immune response that leads to the production of ACTH, MSH, and BE are all highly wavelength dependent. Therefore, action spectra can be used to determine the wavelengths of light that can provide maximum efficiency per photon, and can provide guidance for maximizing efficacy for a targeted UVB phototherapy treatment of autoimmune conditions.

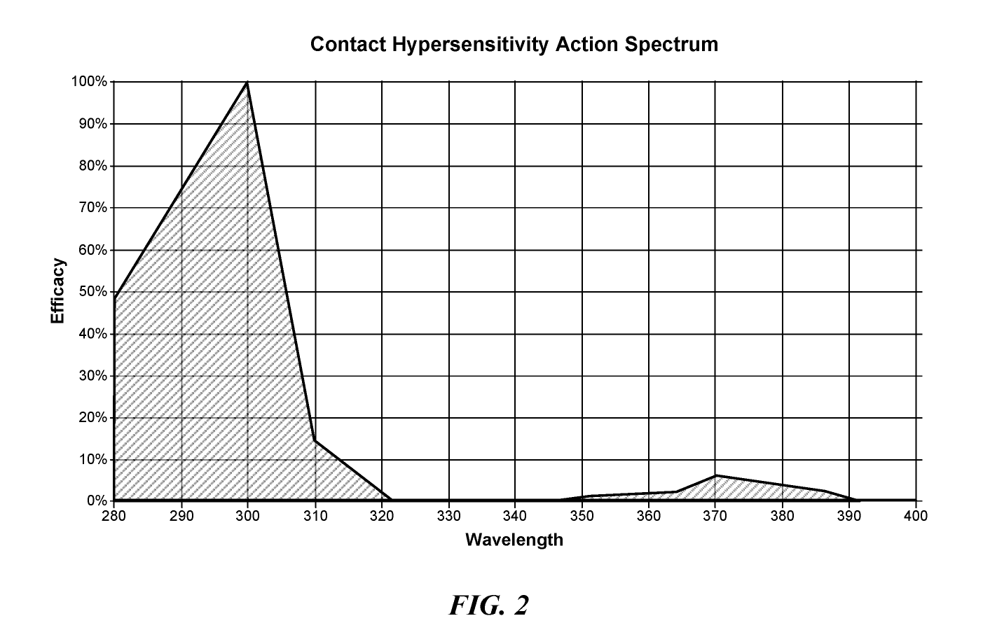

The relative wavelength effectiveness (i.e., action spectrum) has been determined for several indicators of systemic immune response to cutaneous UVB exposure. For example, FIG. 2 illustrates the in vivo action spectrum for the induction of systemic suppression of contact hypersensitivity (a measure of systemic immune alteration).

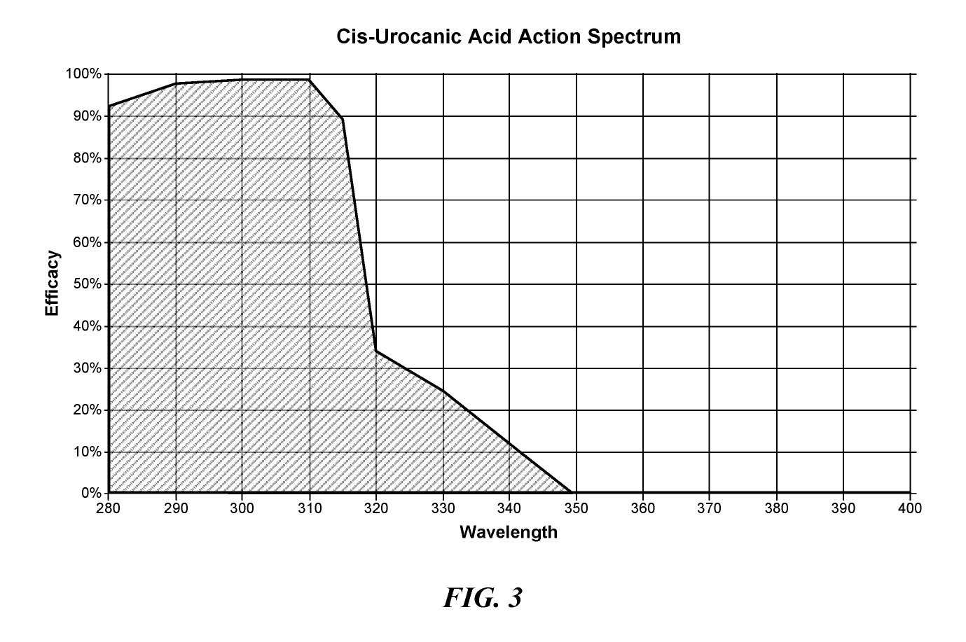

Cis-urocanic acid is a sunlight-induced systemic immunosuppressive factor that has been demonstrated to have a positive impact on UC and MS. FIG. 3 illustrates an action spectrum for cis-urocanic acid production in human skin, and shows a peak in the UVB spectral region of 290-310 nm.

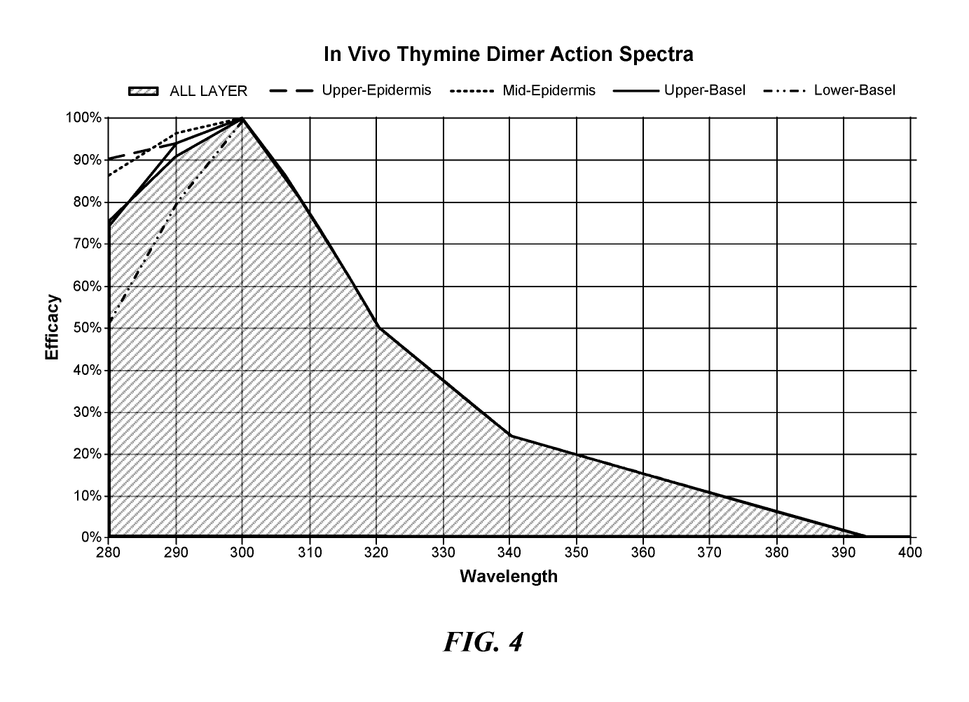

Ultraviolet light causes direct DNA damage in the form of pyrimidine dimers and (6-4) photoproducts, which induce apoptosis of keratinocytes. This activates antioxidant DNA repair enzymes, as well as systemic immune suppression. The in vitro action spectrum for the formation of thymine dimers and (6-4) photoproducts in DNA shows a peak near 260 nm. However, the in vivo action spectrum for epidermal thymine dimer formation shows a peak at 300 nm for all skin layers. The longer peak wavelength is thought to be caused by the significant reduction in epidermis transmission of UV wavelengths shorter than 300 nm. FIG. 4 illustrates an average in vivo thymine dimer action spectrum based on dimer formation for all skin layers tested in the study.

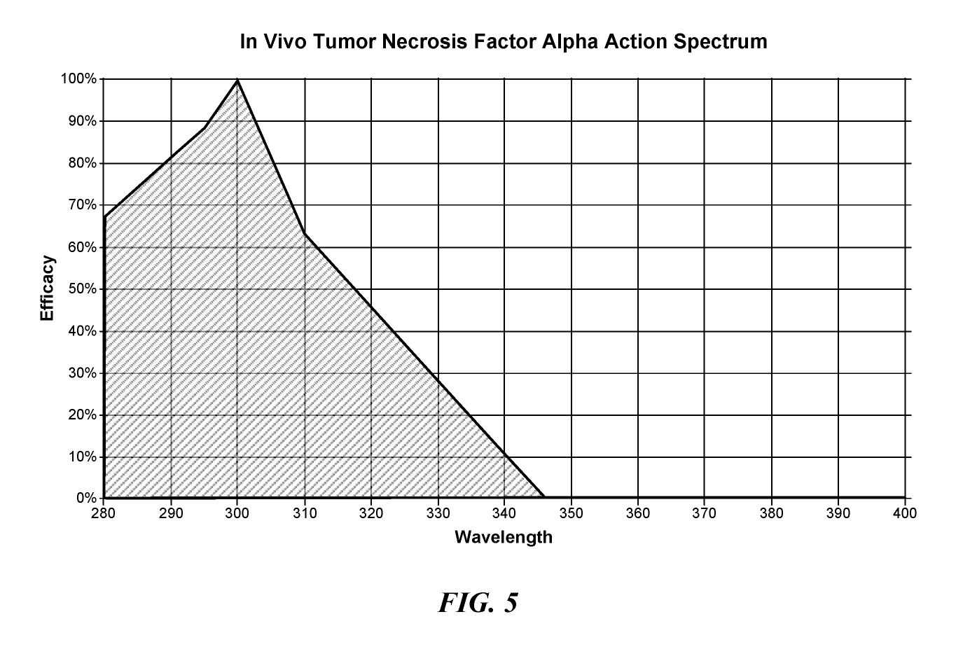

Tumor necrosis factor alpha (TNF) has been found to be an important initiator of the cytokine profile change seen in the skin after UV exposure that favors anti-inflammatory response. It has been shown that TNF serum concentrations can be raised with UVB, thereby influencing the systemic immune system FIG. 5 illustrates an action spectrum for in vivo production of tumor necrosis factor alpha.

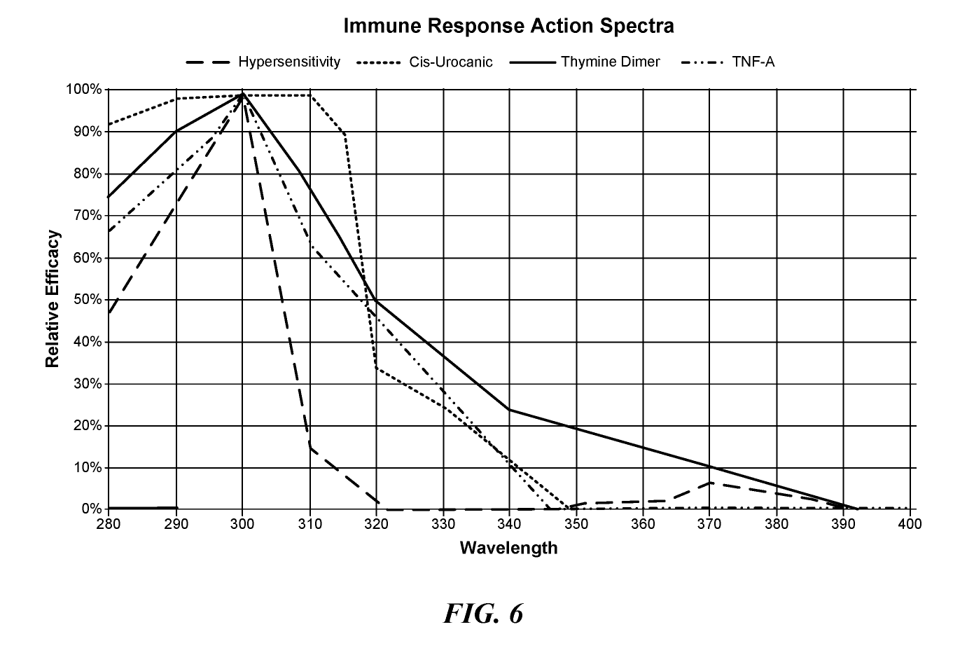

As shown in the graph of FIG. 6, the action spectra for systemic immune response favoring anti-inflammatory immune suppression all have a peak near 300 nm

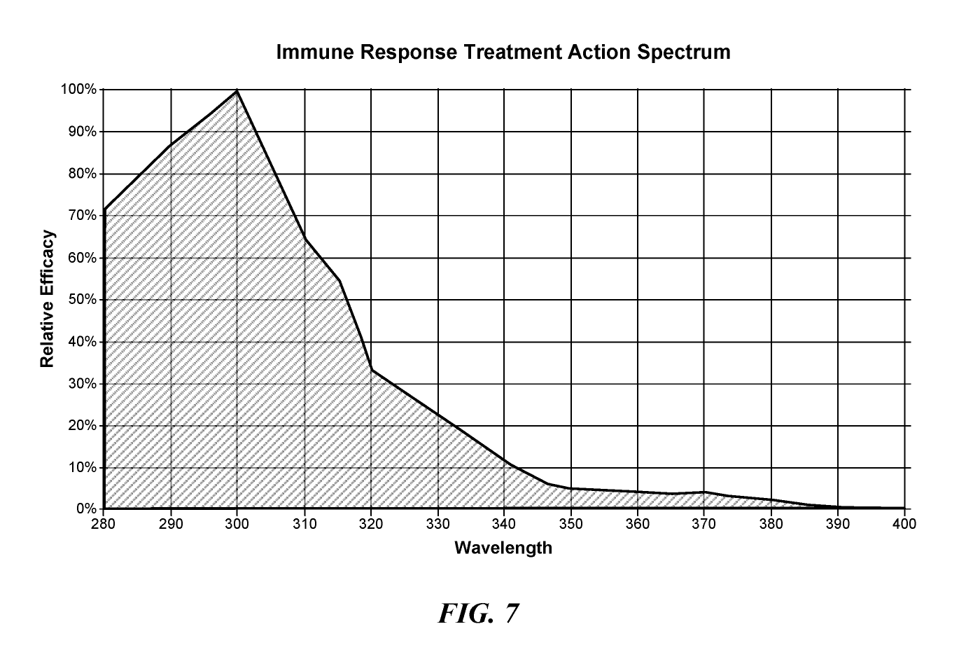

To unify the expression of multiple established action spectra for systemic immune response, the graph of FIG. 7 has been created to illustrate a single action spectrum for immune response treatment of autoimmune disorders. This single action spectrum represents the average efficacy for suppression of contact hypersensitivity, cis-urocanic acid production, all skin layer thymine dimer formation and tumor necrosis factor alpha production at each wavelength of irradiance. The resultant combination action spectrum demonstrates the wavelengths of light that are most effective to elicit systemic immune response needed to treat immune-mediated disorders with minimum total irradiance per phototherapy treatment.

### Vitamin D₃

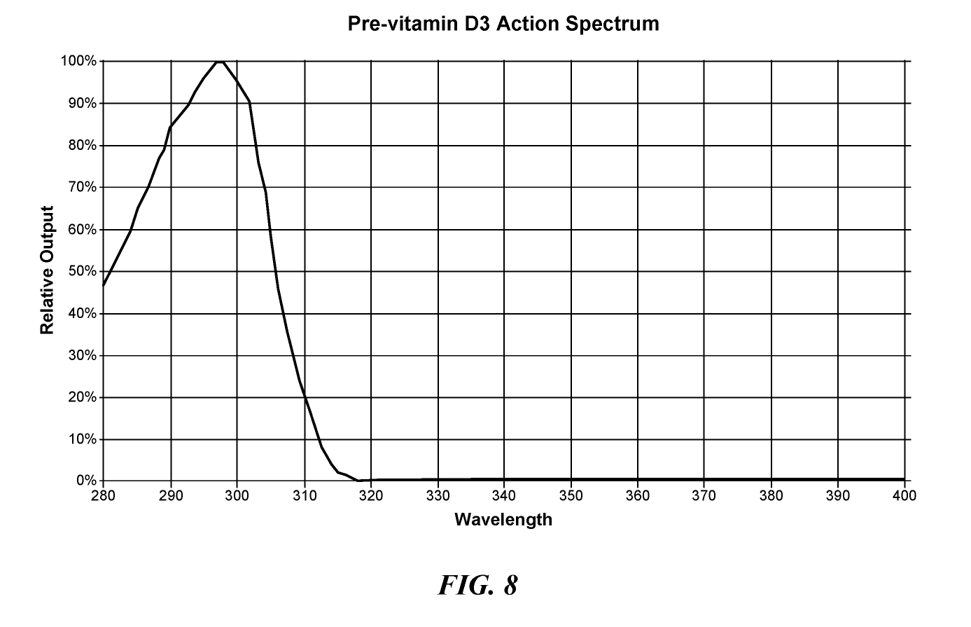

When human skin is exposed to UVB light (280-315 nm) it converts 7-dehydrocholesterol (7-DHC) to pre-vitamin D3 (as well as two other biologically inert photoproducts that regulate production). Pre-vitamin D3 is converted to vitamin D3 in the skin and then transferred to the blood stream over the course of several days. These internal controls result in a deliberately regulated, slow and steady trickle of vitamin D3 to the liver, lasting more than two weeks. After arriving in the liver, vitamin D3 requires two metabolic conversions, (25-hydroxylation in the liver and then 1alpha-hydroxylation in the kidney), to become the active pro-steroid hormone calcitriol. FIG. 8 illustrates a monochromatic UV action spectrum for the conversion of 7-DHC to pre-vitamin D3 in human skin and shows that peak synthesis occurs at 297-298 nm. The same data was further defined and extended by the International Commission on Illumination (“CIE”).

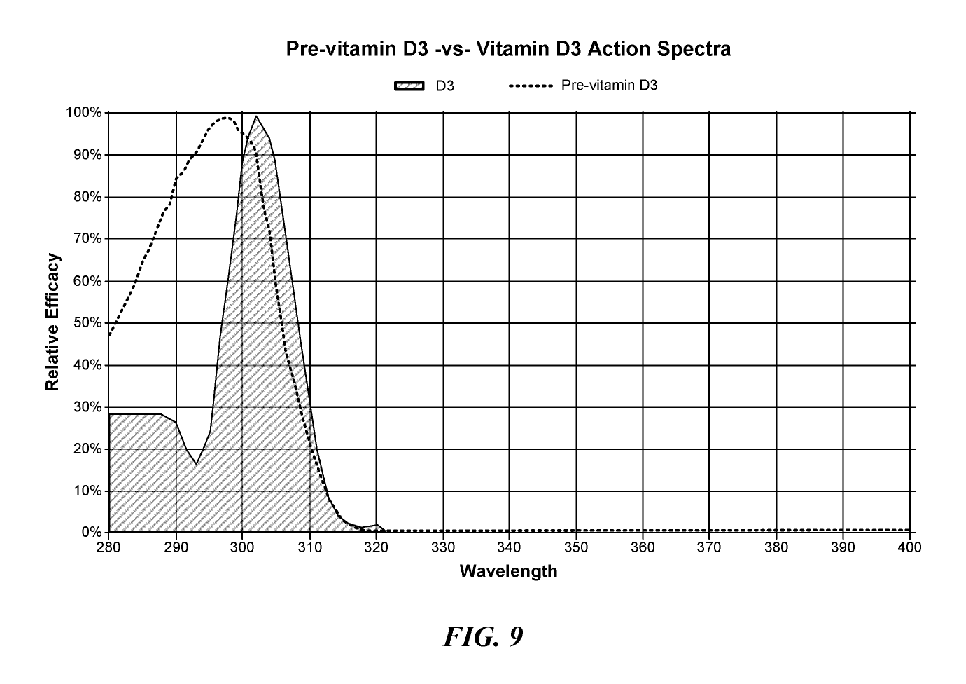

A vitamin D₃ action spectrum was constructed using human skin equivalent exposed to therapeutic doses of UV, showing a peak at 302 nm. The comparison between the pre-vitamin D. and vitamin D₃ action spectra is shown in FIG. 9.

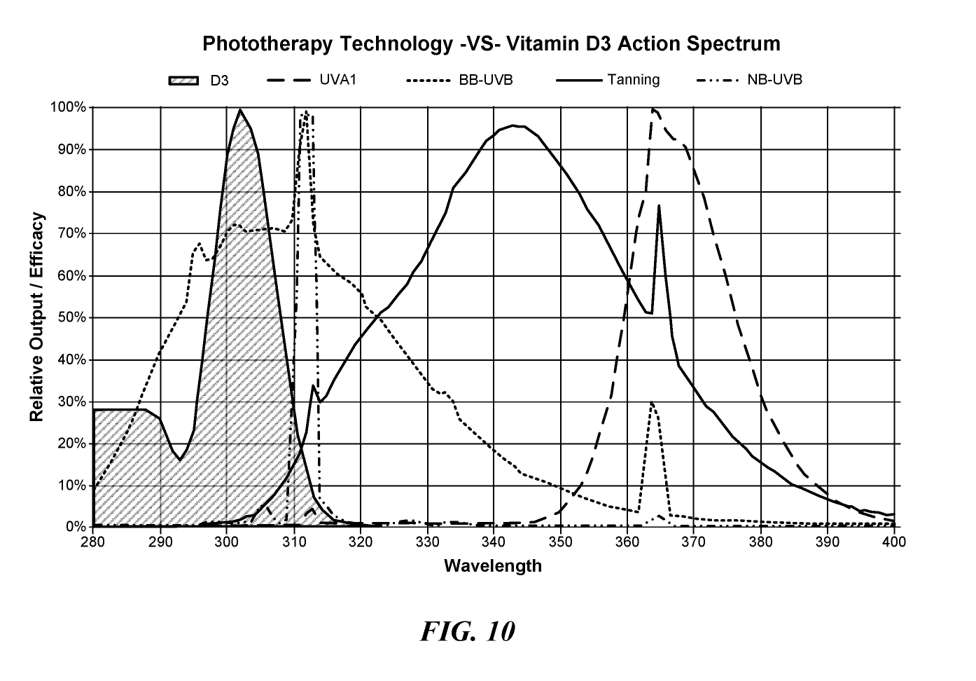

In FIG. 10, the comparison between the vitamin D₃ action spectrum and spectral analysis of four common forms of phototherapy (BB-UVB, NB-UVB, UVA, and tanning) indicates that each phototherapy technology has a different propensity to produce vitamin D₃. Indeed, only phototherapy using UVB can produce significant alteration to serum vitamin D concentration as the UVA spectrum is outside the pre-vitamin D₃ action spectrum. Furthermore, because a larger amount of energy in BB-UVB is within the most effective range of the pre-vitamin D₃ action spectrum, BB-UVB produces more vitamin D than NB-UVB. However, none of the current phototherapy technologies optimize vitamin D₃ production.

### Cutaneous Calcitriol Production

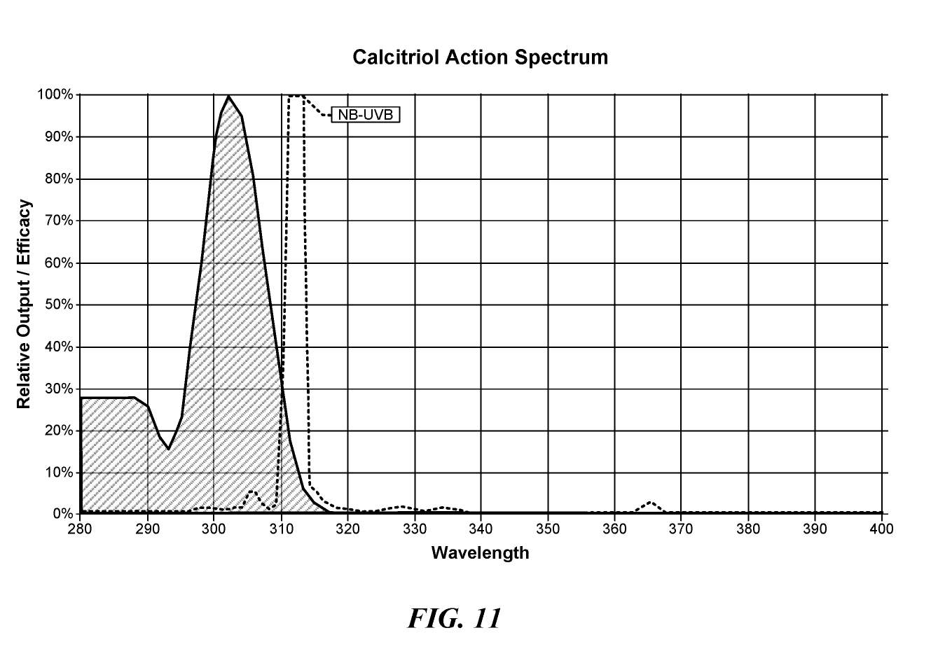

Vitamin D from cutaneous synthesis or dietary intake is sequentially converted in the liver to 25-hydroxyvitamin D₃ and then in the kidneys to calcitriol. However, it has been shown that in addition to this internal process, calcitriol is produced directly in human skin exposed to UVB. Calcitriol photoproduction in the skin is highly sensitive to wavelength, similar to vitamin D₃, with studies demonstrating maximized formation between 300 nm and 305 nm. In fact, the amount of vitamin D₃ photoproduction in the skin directly determines the amount of subsequent calcitriol conversion in the skin. The same study that constructed the vitamin D; action spectrum also determined that the action spectrum for subsequent calcitriol production is identical (FIG. 11).

A narrowband TL-01 lamp made by Philips of Andover, Mass., which is commonly used UVB source for phototherapy, has maximum spectral irradiance at around 311 nm. As shown in FIG. 11, the spectral curve of the TL-01 NB-UVB lamp does not overlap much with the calcitriol action spectrum. Thus, while the TL-01 lamp can produce a small amount of calcitriol, it has been proven that UVB energy at 300 nm (+/−2.5 nm) is significantly more effective at producing calcitriol (e.g., 38 times more effective).

### Erythema & MED

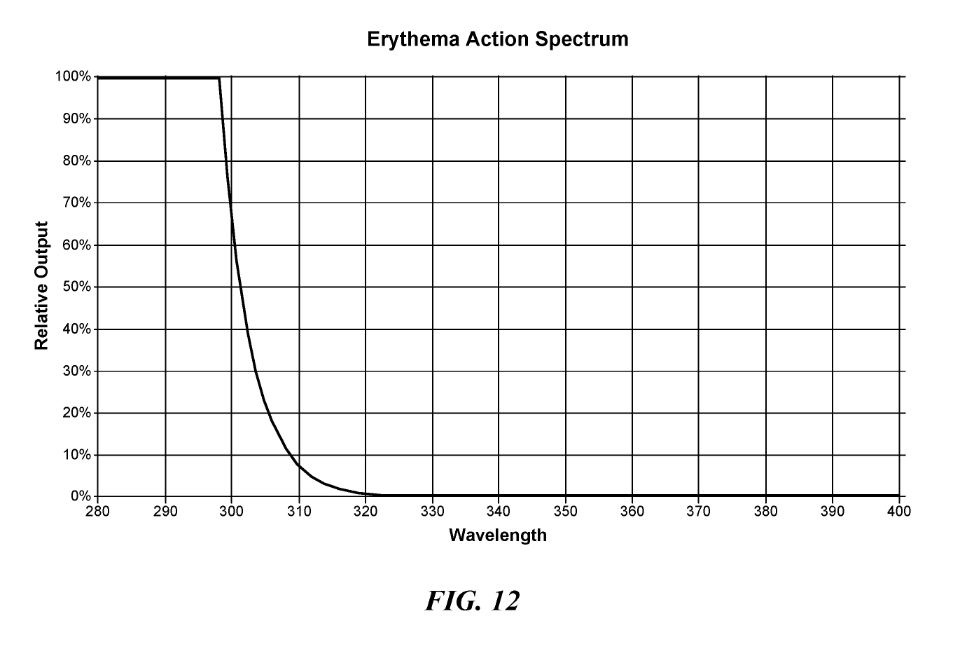

Erythema is redness of the skin caused by increased blood flow which occurs with skin injury, infection, or inflammation. Erythema caused by UV exposure is commonly referred to as sunburn. The Erythema Reference Action Spectrum and Standard Erythema Dose (“SED”), internationally recognized standard published by the CIE (ISO 17166:1999), is used to determine erythema response to individual wavelengths from 250 nm to 400 nm. The CIE action spectrum for erythema is used as a weighting factor for spectral irradiance output from a UV source used for phototherapy treatment. As shown in FIG. 12, the erythema action spectrum has a constant maximum from 250 nm to 298 nm, falls off rapidly between 298 nm and 325 nm, then declines slowly and steadily thereafter.

Standardized ultraviolet doses used in phototherapy treatment are based on the individual patient's Minimal Erythemal Dose (MED) for a given light source. The amount of erythemally weighted UV radiation necessary to produce a slight pink coloration of the skin within 24 hours is called 1 MED. The erythema response of skin to UV radiation is correlated to constitutional skin color which is determined by melanin content. Individuals with darker skin color have more melanin absorbing UVB photons. Therefore dark skin requires more erythemally weighted UV than light skin to achieve a standard MED dosage. Historically, phototherapy applications have used the Fitzpatrick Skin Type classification system to place the constitutive skin color of a patient into one of six classes. According to the Fitzpatrick system, skin type I has the lightest skin color (lowest melanin content) and skin type 6 has the darkest skin color (highest melanin content).

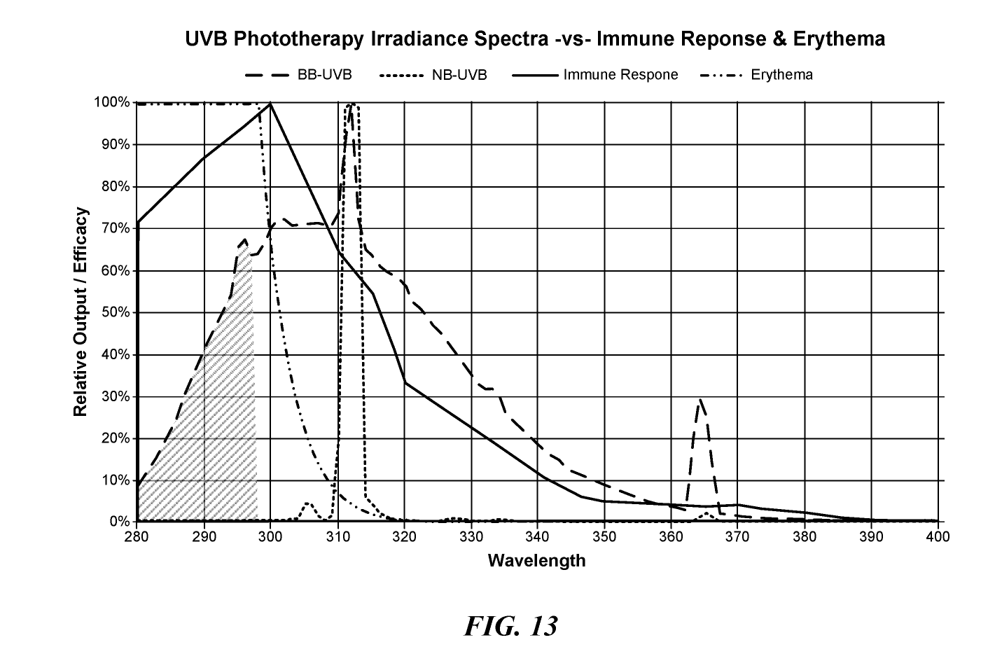

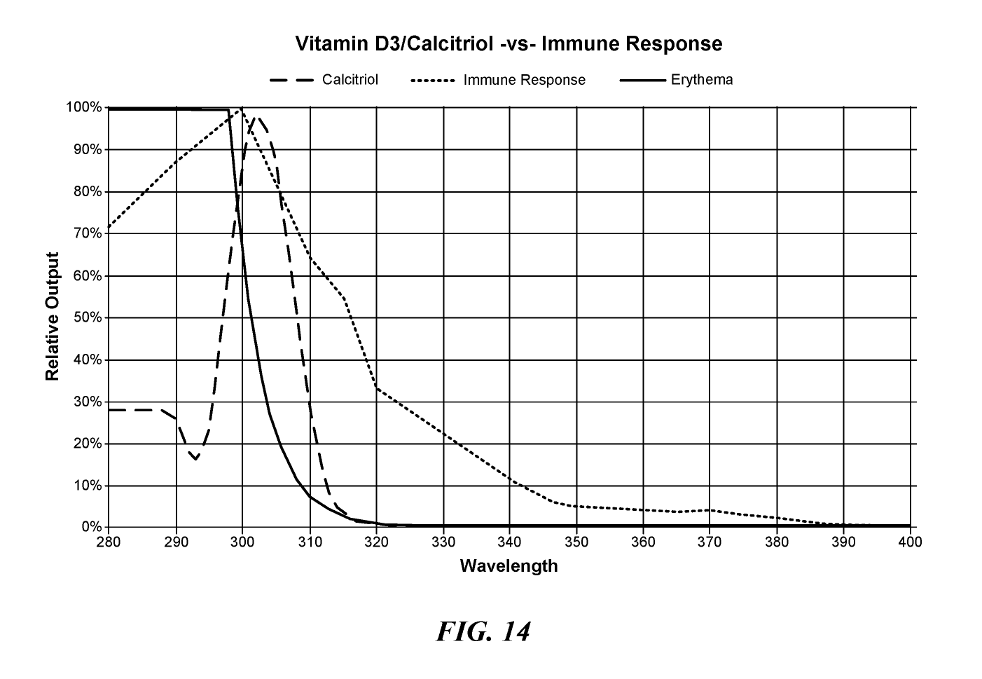

The relationship between erythema and immune response at each wavelength can be important for determining the most effective UV source for autoimmune phototherapy treatment. Specifically, the spectral irradiance of a UV source should deliver energy in a range of wavelengths where the ratio between erythema and immune response is less than 1. Therefore, delivering UV energy with wavelengths shorter than 298 nm would provide progressively diminished therapeutic benefit because the wavelength is reduced to levels below maximum immune response (e.g., approximately 300 nm as shown in FIG. 7), while erythema remains at a constant maximum. FIG. 13 indicates that most of the spectral energy from narrowband UVB (NB-UVB) has an erythema/immune response ratio less than 1 while broadband UVB (BB-UVB) contains significant energy that contributes to erythema more than immune response (i.e., see shaded area of FIG. 14). Thus, FIG. 14 indicates that more total UV energy with greater immune response can be delivered per standardized MED treatment using NB-UVB rather than BB-UVB. Consequently, it has been found that NB-UVB is more effective at treating psoriasis than BB-UVB.

### Combination Action Spectrum

The erythema, pre-vitamin D₃, vitamin D₃, calcitriol and several immune response action spectra have been defined and, as shown in the Figures, are very similar to each other. In FIG. 14, the action spectrum for vitamin D₃/calcitriol photoproduction is shown in comparison to the erythema action spectrum and an action spectrum constructed from multiple immune response spectra (i.e., the immune response treatment action spectrum of FIG. 7).

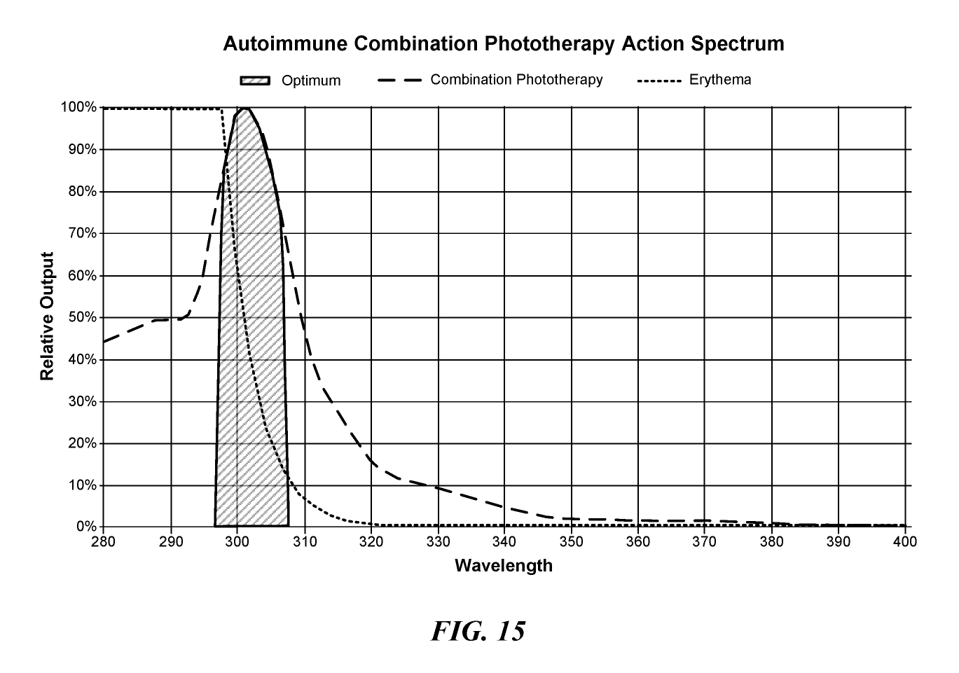

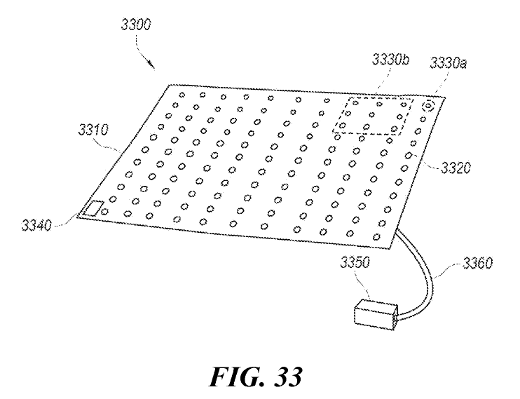

FIG. 15 illustrates a “combination phototherapy action spectrum”, that includes the average of the vitamin D3/calcitriol action spectrum of FIGS. 9 and 11 and the previously constructed immune response action spectrum of FIG. 7. This combination action spectrum expresses the maximum efficacy for both immune response and vitamin D₃/calcitriol production in the skin. A device that isolates and delivers to the skin a UV spectrum maximizing calcitriol production and immune response under the action spectrum of FIG. 15 is expected to provide the most efficacious phototherapy treatment of autoimmune disorders. As shown in FIG. 15, the optimal wavelength range for maximum phototherapy efficacy is between 298 nm and 307 nm, with minimal UV energy at wavelengths shorter than 298 nm or longer than 307 nm. Accordingly, a phototherapy device, such as those described in further detail below with reference to FIGS. 32 and 33, that emits more than 75% of total UV output within the wavelength range 298 nm to 307 nm is expected to be most effective and safe for the treatment of autoimmune disorders.

### Treatment Dosage

Phototherapy dosage can be described as the product of the intensity (or irradiance) of a light source and the time of exposure to that light source (Dose=Intensity×Time). Therefore, a desired dosage may be achieved by increasing or decreasing the intensity of the radiation source and/or exposure time. Dosage can be expressed in millijoules per centimeter squared (mJ/cm²) when intensity (or irradiance) is expressed in milliwatts per centimeter squared (mW/cm²) and time is expressed in seconds. As explained in further detail below, for phototherapy applications, several additional factors can influence the calculation of intensity and dosage for a particular radiation source and configuration of that source relative to the patient.

### Dosage and Selected Embodiments of Phototherapy Systems and Devices

Phototherapy can be delivered to the skin with systems that provide a substantially uniform distribution of energy to the treatment area of the skin, and the uniformity with which the phototherapy is applied can affect the dosage level delivered during a phototherapy session. More specifically, the dosage delivered to the entire treatment area is limited by the largest dosage level applied to any one area of the skin. For example, if a treatment area is 100 cm² and the phototherapy system used to deliver the phototherapy to the treatment area has a non-uniform energy distribution that exposes 10 cm² of the treatment area to twice the intensity as the intensity applied to the other 90 cm², the dosage of the entire treatment area is limited by the maximum dosage that can be applied to the 10 cm² treatment area This results in 90 cm² of the treatment area being exposed to half of the maximum or desired dosage. Accordingly, phototherapy systems that emit radiation with greater uniformity are expected to enhance treatment efficacy.

Various mechanisms can be used to emit and apply the irradiance of a light source or system of light sources to the skin with relative uniformity. In certain embodiments, a phototherapy device includes one or more low-energy radiation sources (e.g., 3 Watts or less) that can be positioned in close proximity to the treatment area on the patient (e.g., 3 cm or less). This allows the phototherapy to be delivered to selective and scalable treatment areas. In other embodiments, the phototherapy device includes one or more high-energy radiation sources (e.g., 25 Watts or greater) that are spaced apart from the treatment area on the patient by a distance large enough (e.g., 10 cm or more) to allow distribution of the emitted energy from the radiation sources. For example, the radiation sources may have an emission pattern that has an uneven distribution of intensity at a position close to the radiation source (e.g., a higher intensity at the center of the emission pattern), but that distributes light outwardly such that the radiation source provides a substantially uniform distribution of radiation intensity when spaced further from the radiation source. In this embodiment, the phototherapy can be applied over a large treatment area (e.g., 100 cm² or greater).

The low-energy phototherapy system can include one or more small, radiation sources with relatively monochromatic wavelength emissions. These radiation sources can be configured such that they do not require a separate filtering method (e.g., a coating) and may be assembled in tightly-packed arrays. For example, the radiation source can be a light emitting diode (LED). In a phototherapy system using LEDs as the radiation source, the LEDs can be configured to emit radiation at a specific wavelength target with most of the optical energy emitted within a small bandwidth (e.g., a 10 nm bandwidth) suitable for phototherapeutic treatment of autoimmune disorders, dermatological disorders, vitamin D phototherapy, and/or other indications. For example, the wavelengths of the LEDs can be selected using the methods described above with respect to FIGS. 1-16. In certain embodiments, the LEDs can emit wavelengths between 298 nm and 307 nm. In other embodiments, the LEDs can have one or more different wavelengths, such as wavelengths ranging from 295 nm to 310 nm. The individual LEDs can also include one or more lenses or other features that diffuse or otherwise spread the emitted light at least substantially evenly across a surface area. A larger lens can be used in addition or as an alternative to the individual LED lenses, and placed over more than one LED to enhance the uniformity of emissions across several LEDs. In various embodiments, the LEDs of the phototherapy system are arranged in tightly packed arrays, such as arrays of 50 or more LEDs. The intensity of the LED array can be selected by adjusting various parameters of the array and associated components. For example, the intensity of the LED array can be increased by increasing the input energy delivered to the LEDs (e.g., by changing the power source or controls thereon), increasing the quantity of LEDs per unit area, decreasing the distance between the LEDs and the treatment area on the patient (e.g., 0-3 cm), decreasing the degree of light spreading of the lens(es) on the LEDs, and/or changing other features of the LED array that impact the radiation intensity. Conversely, the intensity of the LED array can be decreased by decreasing the level of energy delivered to the LEDs, decreasing the quantity of LEDs per unit area, increasing the distance between the LEDs and the treatment area on the patient, increasing the degree of light spreading of the lens(es) on the LEDs, and/or changing other features of the LED array that impact the radiation intensity.

The LED-based phototherapy system can provide an at least substantially uniform distribution of irradiation intensity by taking into account various features of the system, such as the distance between the LEDs and the treatment site on the patient, the spacing of the LEDs with respect to each other, and/or the shape of the lenses on the individual LEDs. For example, the LED array can be arranged such that at least a major portion of emission patters of the individual LEDs do not overlap each other such that irradiation from one LED of the array does not overlap the irradiation of another LED. The lenses on the individual LEDs can be used to expand or contract the LED emissions of the individual LEDs such that they do not overlap each other. In certain embodiments, LEDs are spaced apart by a distance that avoids overlapping LED emissions, but also leaves some portions of the treatment area (e.g., the area opposed to the area of the LED array or the area within emission area of the LED array) unexposed from the LED emissions. For example, the LEDs may be spaced apart by a distance such that 20% of the treatment area is not exposed to the LED emissions while the remaining 80% of the treatment area is exposed to a substantially uniform level of intensity from the LEDs. In other embodiments, the LEDs are spaced apart in such a manner that 30%, 40%, or 50% of the treatment area of the patient is unexposed, while the corresponding 70%, 60%, or 50% of the treatment area is exposed to a substantially uniform level of intensity.

When the LED-based phototherapy system is configured to provide an at least substantially uniform irradiation intensity, it is important that the LED array remain at a constant distance from the treatment area during the phototherapy session to maintain the uniform exposure to the phototherapy source. Accordingly, in certain embodiments the phototherapy device is designed to come in direct contact with the treatment area (e.g., the radiation source is placed on the patient's skin). For example, the phototherapy device can include a sensor that indicates when the device is appropriately placed on the skin to confirm direct skin contact before and/or during operation of the device during a phototherapy session. The phototherapy device can include a strap, an adhesive, and/or another type of fastener that allows the LED array to attach directly to the treatment area. In these embodiments, the constant distance from the skin surface to the radiation source is maintained by the device design itself, rather than being subject to movement of the patient or operator discretion.

Low-energy phototherapy devices, such as the LED-based device described above, can be a wearable device that can be attached to the patient or positioned immediately adjacent to the patient's skin. The wearable phototherapy device can include a radiation source (e.g., an LED array) affixed to a substrate, such as a flexible or non-flexible sheet or fabric that can carry the radiation source. The wearable phototherapy device can take the form of a pad or mat on which the patient can lay, sit, or stand, a patch that can be adhered to a patient's skin, panel incorporated into an article of clothing or other wearable item, a blanket, a cuff, a cap, a shirt, a jacket, pants (e.g., leggings), a sock, a glove, a vest, a cape, a watch, a wand, a paddle, a comb, and/or other suitable items that can be applied directly to the patient's skin. The wearable phototherapy device can be constructed to provide a substantially uniform and constant level of radiation intensity across the portion of the device including the radiation sources. In various embodiments, the wearable phototherapy device can also allow for adjustments in the dosage by altering input energy through system controls and/or time of exposure.

High-energy phototherapy systems can include one or more radiation sources that emit a large amount of energy in the selected UVB range (e.g., 298 nm-307 nm) and a filtration mechanism that blocks unwanted wavelengths outside of the selected range. The radiation source can include one or more mercury arc lamps, pulse and flash xenon lamps, fluorescent lamps, metal halide lamps, halogen lights, and/or other suitable radiation sources for phototherapy. The phototherapy apparatus can include a plurality of radiation sources, such as 5 lamps, 10 lamps, 20 lamps, 30 lamps, 40 lamps, 50 lamps, or more depending on the type of lamp, the desired size of the treatment area, the desired intensity, and the desired phototherapy time. In certain embodiments, the radiation source itself can include filtration mechanisms. In other embodiments, the phototherapy system includes additional filtering features separate from the radiation source to emit the desired wavelength range. The filtration mechanism can include absorption filters and/or interference filters. The high-energy phototherapy system can provide an at least substantially uniform distribution of irradiation intensity by taking into account various features of the system, such as the distance between the radiation sources (e.g., no overlapping emission patterns), the shape of any lenses on the radiation sources, and the distance the patient must be positioned away from the radiation sources to receive substantially uniform irradiation distribution. In addition, the output of the phototherapy system may be adjusted by changing the energy input, the number of lamps, lens specifications, and/or filtration parameters.

### Intensity (or Irradiance)

The intensity of a radiation source can be measured as the absolute milliwatts per centimeter squared (mW/cm²) measured at a given distance from the source. As the distance between the source and measurement position increases the intensity of the measurement will decrease. In high-energy phototherapy devices, the intensity of a phototherapy device is assumed to be measured at the position of the patient relative to the radiation source. If the distance from the radiation source to the patient varies greatly between patients, between phototherapy sessions, or along the body of single patient, the uniformity and intensity of the irradiance becomes too varied for consistent dosages of phototherapy applications. Accordingly, in various embodiments, phototherapeutic devices can be configured such that the distance of the patient from the radiation source is assumed to be no less than 10 cm and no greater than 200 cm. Within this range, a standard position for the patient can be determined for a phototherapy device configuration such that the variation of the patient's position is no greater than about 25% of the total distance of the lamp source to the patient (e.g. 2.5 cm-50 cm). In low-energy devices, the radiation source is assumed to be directly in contact with the patient's skin, or at no greater distance from the treatment site than 3 cm.

Intensity for a radiation source in phototherapy applications uses “The Erythema Reference Action Spectrum” (ISO 17166:1999) as a weighting factor for spectral irradiance output measurement. The absolute measured intensity (mW/cm²) for each wavelength is multiplied by the weighting factor for that wavelength to determine the erythemally weighted irradiance. The sum of all erythemally weighted irradiance for each individual wavelength equals the total erythemally weighted irradiance (or intensity) for the phototherapy device. This erythemally weighted intensity can be used in calculations related to dosage (Dose=Intensity×Time). According to the ISO standard, 1 Standard Erythema Dose (SED) is equivalent to an erythemal effective radiant exposure (EERE) of 10 mJ/cm². Radiation sources that have the same absolute intensity can have a significant difference in exposure time needed to achieve 1 SED, even within the relatively narrow optimal wavelength range for maximum phototherapy efficacy (e.g., 298 nm-307 nm) because of the weighting factor applied to the absolute measured intensity of each wavelength. In various embodiments, phototherapy systems (e.g., the phototherapy systems described with references to FIGS. 32-35 below) can be configured to expose a user to less than 10 SED of radiation during a phototherapy session (e.g., 1-10 SED of energy).

### Skin Exposure

Phototherapy treatment of autoimmune disorders can consist of one or more individual treatment sessions using a device that delivers a dose of UV radiation. Because exposure to UV radiation thought to be damaging to skin tissue and may be related to other conditions, safety of a phototherapy session can be increased by reducing or minimizing of total UV exposure. The amount of calcitriol, vitamin D₃, and systemic immune response produced within the UVB range is directly related to the total surface area of the skin exposed during a treatment. Increasing surface area of the skin exposed to UVB will increase all of these responses, thereby increasing treatment efficacy while minimizing total UV exposure to any one area of the body because full body exposure does not require the intensity necessary for “spot treatment” (i.e., exposing only a small targeted area of skin to UVB radiation). The effectiveness of this method can be magnified using a focused UVB range. For example, a phototherapy device that emits the majority of total UV output within the wavelength range 298 nm to 307 nm is consistent with the combination phototherapy action spectrum (FIG. 15) and, therefore, will produce significantly more calcitriol, vitamin D₃, and systemic immune response using significantly less total UV radiation than existing phototherapy technologies. For example, the present technology can distribute this focused energy evenly across a large surface area of the skin to improve efficacy of the treatment, while simultaneously reducing the total UV radiation to any one area.

Improvement to treatment efficacy using focused UV (298 nm-307 nm) can be obtained by maximizing skin surface exposure during each phototherapy session. It is thought that the minimum threshold of skin surface area that needs to be exposed to provide the systemic therapeutic benefit is about 30%. There is thought to be a direct correlation between percentage of skin surface area exposed (30%-100%) during a treatment session and overall treatment efficacy. Exposing at least 30% of a patient's total skin surface area to a focused UV range (298 nm-307 nm) during a single phototherapy session would allow efficacious treatment of autoimmune disorders in various systems of the body including nervous, digestive, endocrine, integumentary, cardiovascular, muscular, and skeletal. This can be accomplished with a high-energy device, which easily treats large surface areas. Low-energy devices can also be configured to include larger arrays of radiation sources to provide for the treatment of large areas (e.g., a mat, jacket, or blanket). Alternatively, low-energy phototherapy devices that are smaller in scale can be used multiple times at various locations on the patient's body during a single phototherapy session (e.g., as in a small pad).

### Defining Dosage

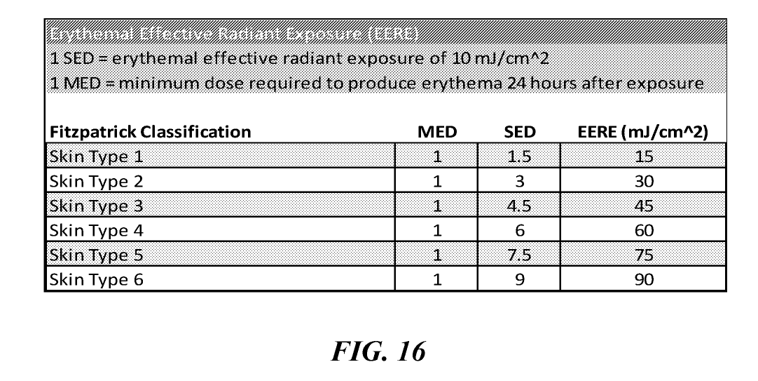

As discussed above, the Standard Erythema Dose (SED) is a standardized measurement of erythemogenic UV radiance density (not be confused with the Minimal Erythema Dose (MED) used in phototherapy treatment). Determining the appropriate dose for treatment is based on the constitutive skin color of the patient, which can be expressed as a Fitzpatrick Skin Type 1-6. Skin type can also be determined by answering a series of questions related to the Fitzpatrick Skin Type scale (e.g., on an automated user interface or manually provided), determined automatically using a sensor or detector that measures the reflectance, absorption, and/or color of a patient's skin, and/or determined using a grid that allows a patient or clinician to match the patient's skin tone to predetermined skin characteristics (e.g., fair, burns quickly; burns moderately, tans easily, etc.) and/or skin images of colors. In other embodiments, the patient's skin type can be determined automatically using other sensors and/or through automated and/or manual questionnaires or charts. The skin type is used to calculate I Minimal Erythema Dose (1 MED) is the amount of Erythemal Effective Radiant Exposure (EERE expressed in mJ/cm²) needed to produce a slight pink coloration of the skin within 24 hours. Because MED takes into consideration the skin type of the patient and the amount of EERE relative to that skin type, a “standard” phototherapy dose can be represented as a decimal of MED for all skin types. For example, a standard phototherapy dose for treatment with a device may be selected to be a constant 0.75 MED (or 75% of I MED) for all skin types. With 0.75 MED as the constant, the amount of EERE (mJ/cm²) becomes a variable that is adjusted according to skin type to achieve 0.75 MED. The exact amount of EERE needed to achieve 1 MED for each skin type (i.e., Skin Types 1-6) is expected to lie between 15 mJ/cm² to 90 mJ/cm², equivalent to 1.5 SED to 9 SED. The relationship between skin type, MED, SED and EERE is reflected in FIG. 16.

Skin type and MED can be determined using an instrument that measures skin reflectance, absorption, and/or color, or with information obtained from a questionnaire. Because skin reflectance instruments must typically come in direct contact with the skin, such instruments can integrated into an LED array as part of a low-energy phototherapy system. In high-energy phototherapy systems, skin reflectance, absorption, and/or color instruments can be incorporated into the system such that skin type and MED can be determined before treatment begins. With both high-energy and low-energy systems, a questionnaire could be administered and skin type determined before the treatment begins.

The UV dose for phototherapy treatment of autoimmune disorders can be selected such that it produces significant efficacy without side effects. A phototherapy device that emits more than 75% of total UV output within the wavelength range 298 nm to 307 nm can be both effective for the treatment of autoimmune disorders and avoid side effects. However, a dosage range is needed to provide guidance for avoiding side effects and providing a high degree of efficacy. Because MED takes several variables into consideration, dosage provided by a phototherapy device can be expressed as a decimal MED constant. For example, a phototherapy device with focused UV range (e.g., 298 nm-307 nm) can have a dosage range of 0.2 MED (20% of I MED) to 0.9 MED (90% of 1 MED). Within this dosage range, 0.2 MED is expected to be least efficient, but also have a relatively lower risk of side effects caused by skin exposure to UV, whereas 0.9 MED is expected to be the most efficient. As dosage is increased, there is an equal increase in the level of UV exposure. Therefore, in certain embodiments the dosage can be selected to have an equal balance of UV exposure and efficacy, such as 0.55 MED. In other embodiments, the dosage can be higher or lower than 0.55 MED depending on the phototherapy device used, the type of efficacy and UV exposure desired, and a patient's skin type.

Combining MED Dosage with Skin Exposure

Combining dosage with skin exposure percentage can be used to adjust the balance of UV exposure and efficacy. As described above, the efficacy of the phototherapy treatment is expected to be a function of, at least in part, the amount of surface area of the patient's skin exposed to UV radiation and the degree of MED applied, with more skin exposure and higher levels of MED expected to provide a more effective therapy. In certain embodiments, for example, the dosage range for a phototherapy treatment session using a focused UV range (298 nm-307 nm) includes a maximum dose of 0.9 MED to 100% of a patient's skin surface area to a minimum dose of 0.2 MED to 30% of a patient's skin surface area Skin exposure percentage contributes to efficacy, but not safety. In other embodiments, more or less of the patient's skin can be exposed and/or more or the MED range can differ.

It is expected that the percentage of skin exposure percentage contributes to efficacy of the phototherapy, but not does not necessarily impact the risk of side effects. For example, if dosage is held constant (e.g., at 0.55 MED) and skin exposure percentage is increased, the efficacy is expected to increase without increasing the risk of side effects. Accordingly, as long as dose is 0.2 MED to 0.9 MED and skin exposure percentage is greater than 30%, it is possible to trade dose and exposure percentage to achieve a desired efficacy and mitigate the risks of potential side effects. That is, phototherapy dosages and the resultant efficacy can be selected based on the total skin exposure (e.g., 30%-100%) and the percentage of 1 MED (e.g., 20%-90%), and these two parameters (i.e., percentage skin exposure and MED dose) can be selected based on the desired result and patient-specific needs (e.g., specific indication, autoimmune disease, skin type, etc.).

It is also possible to maintain a constant efficacy by varying skin exposure percentage relative to MED dosage. Accordingly, increasing the skin surface area exposed can lower the necessary MED dosage to achieve the same level of efficacy. For example, the same efficacy in a phototherapy session can be achieved with a 0.2 MED dose and 100% skin exposure as with a 0.4 MED dose and 50% skin exposure. Similarly, phototherapy treatment sessions can have the same efficacy with (a) a 0.4 MED dose and 60% skin exposure as with a 0.8 MED dose and 30% skin exposure, or (b) a 0.9 MED dose and 40% skin exposure as with a 0.45 MED dose and 80% skin exposure. It is thought that the MED dosage is the parameter that best controls the side effects of the phototherapy session (e.g., exposure to UV radiation), whereas the percentage of skin exposure does not. Therefore, in various embodiments, the selected dosage includes an increased percentage of skin exposure and a decreased MED dosage.

### Dosage Tables

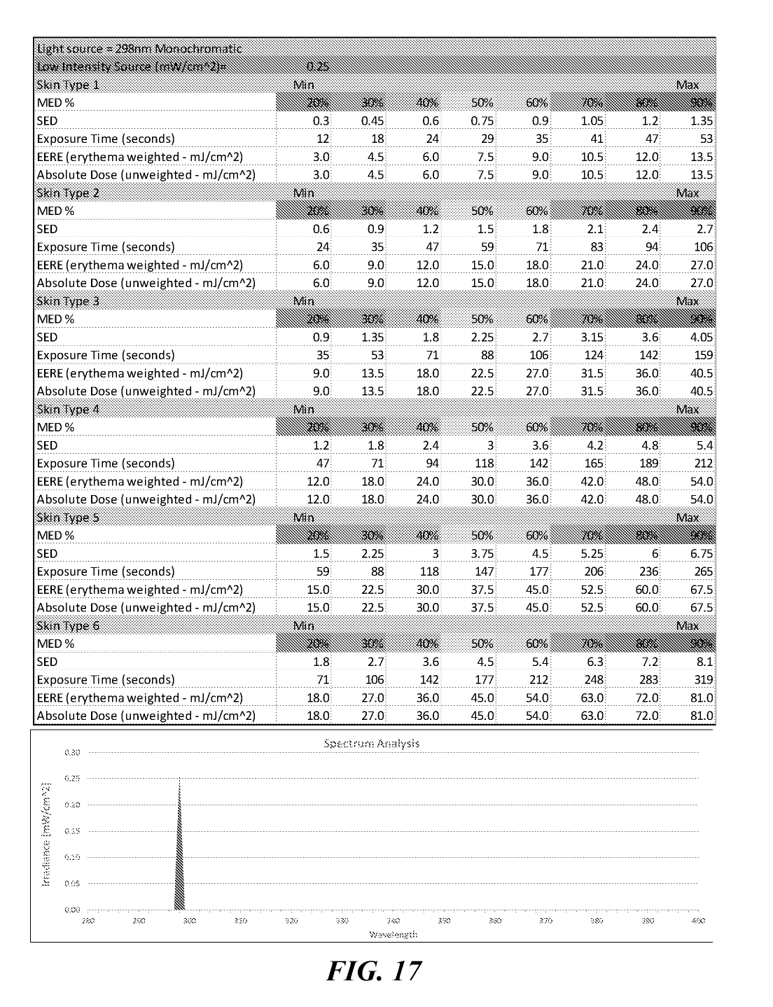

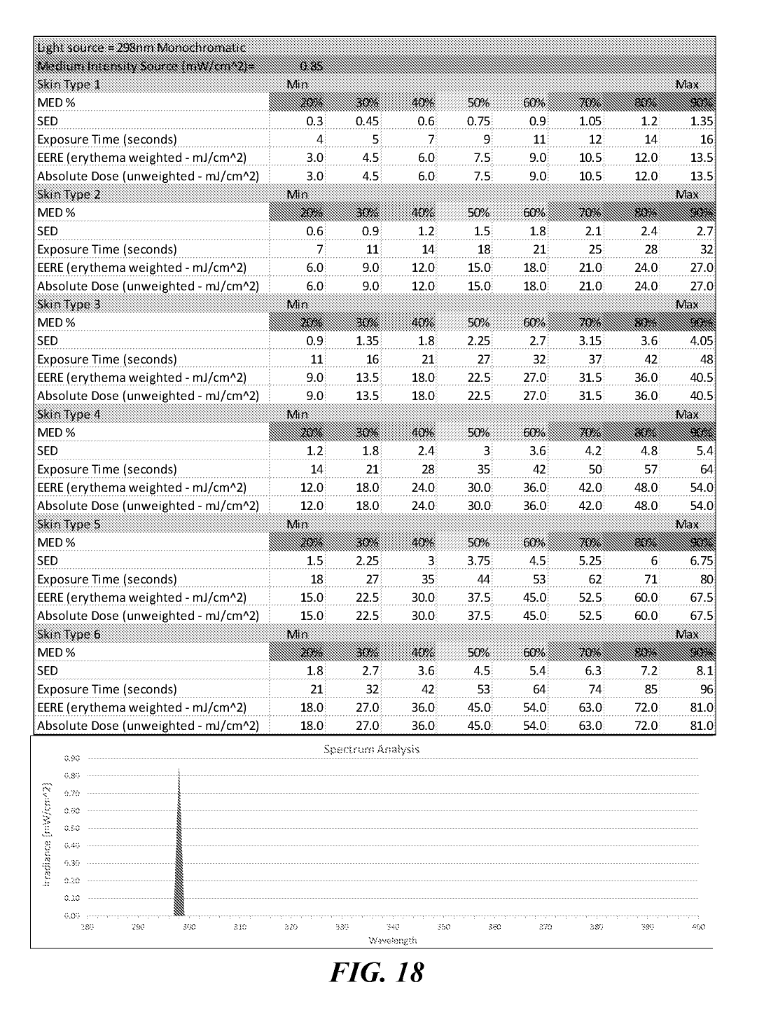

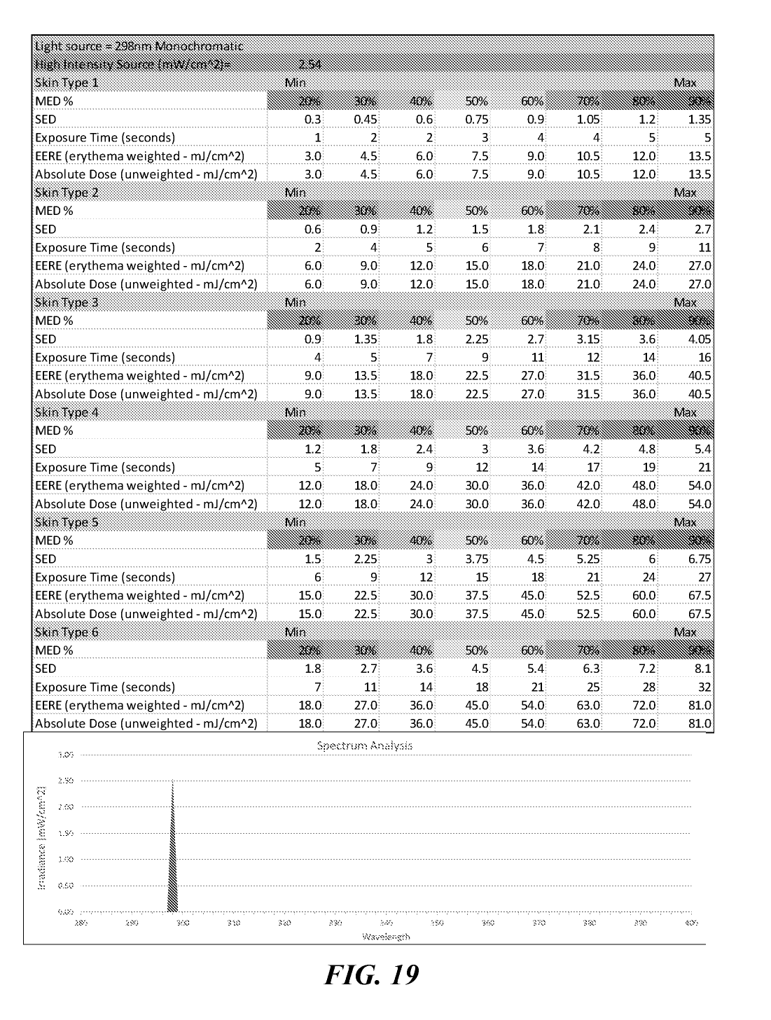

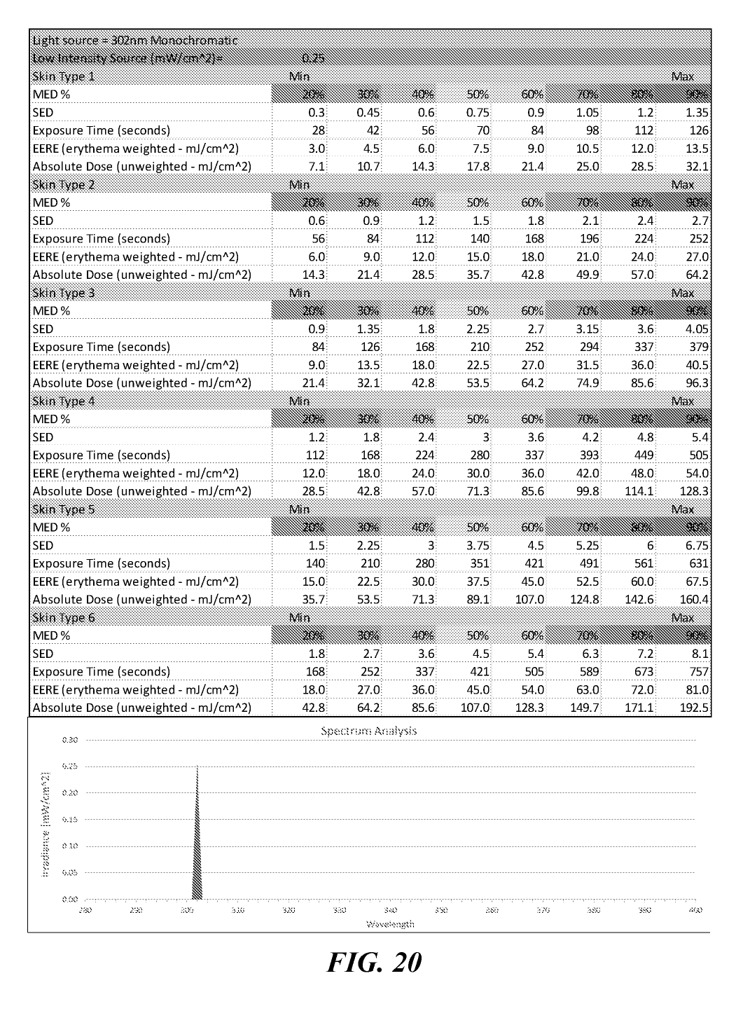

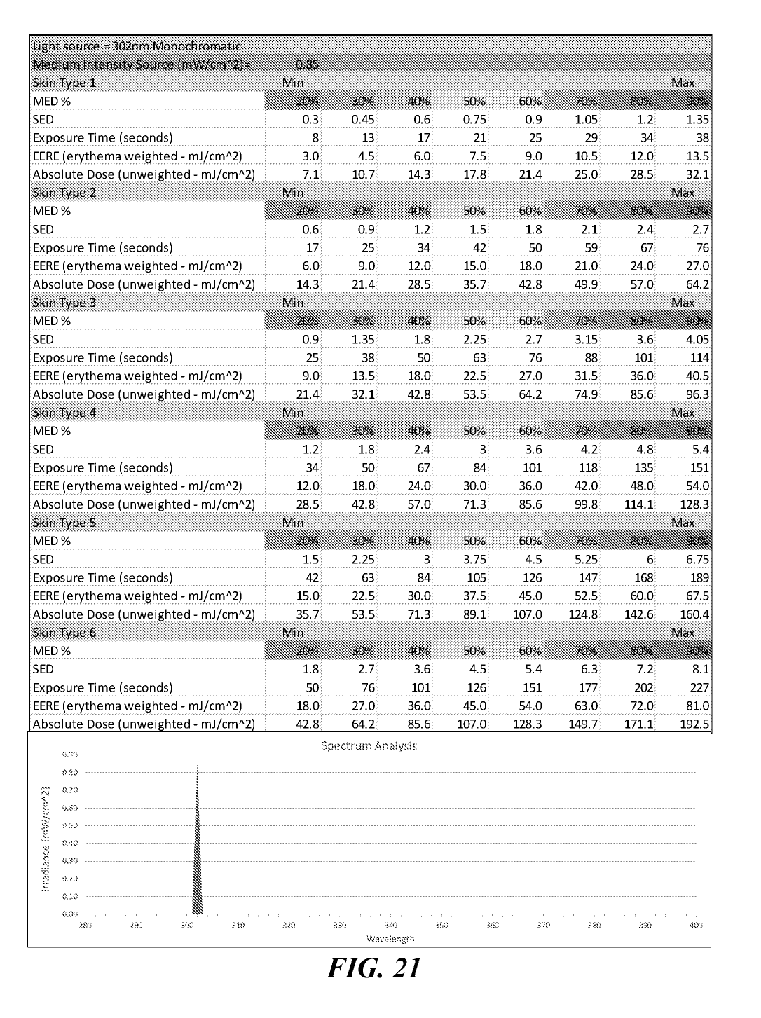

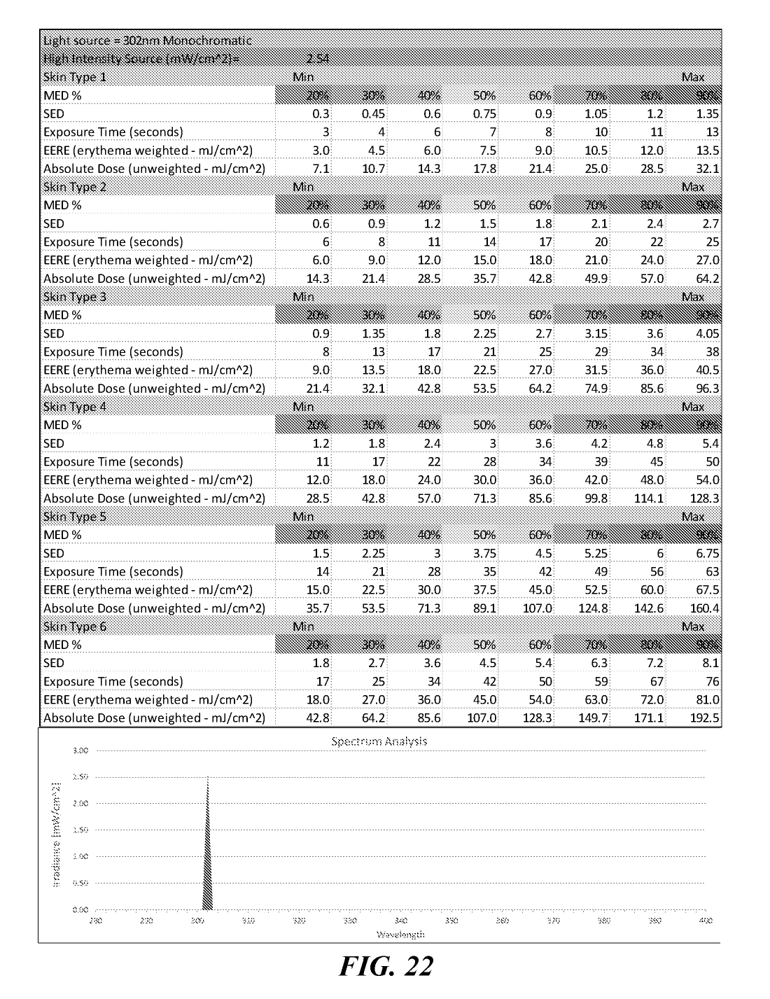

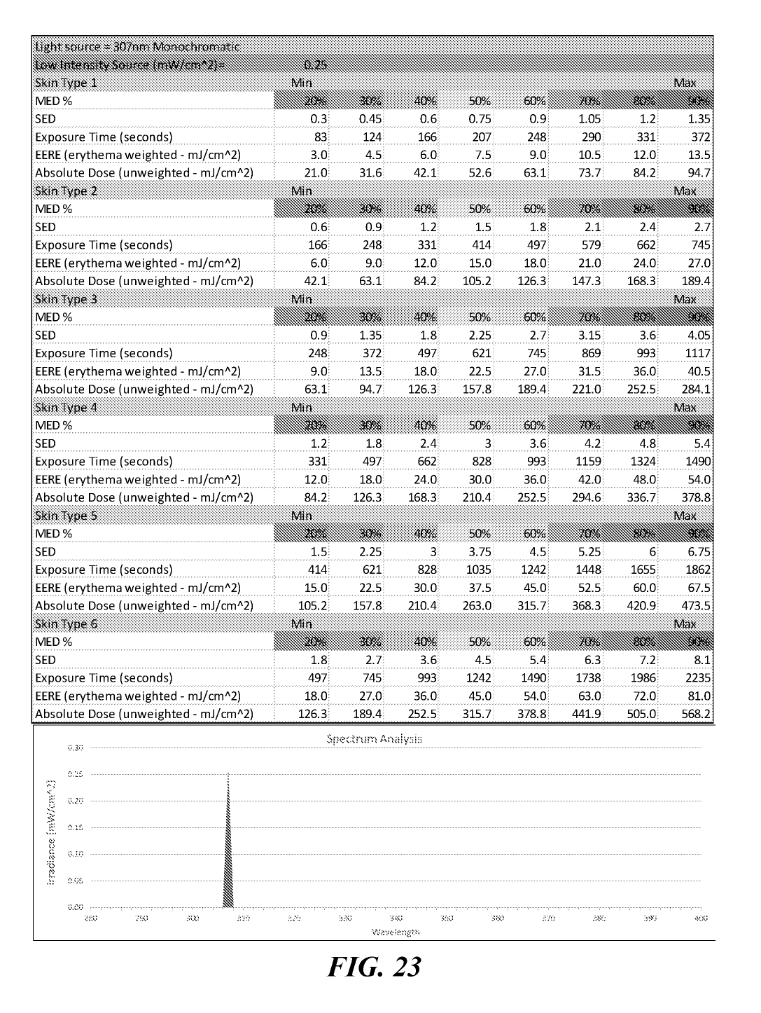

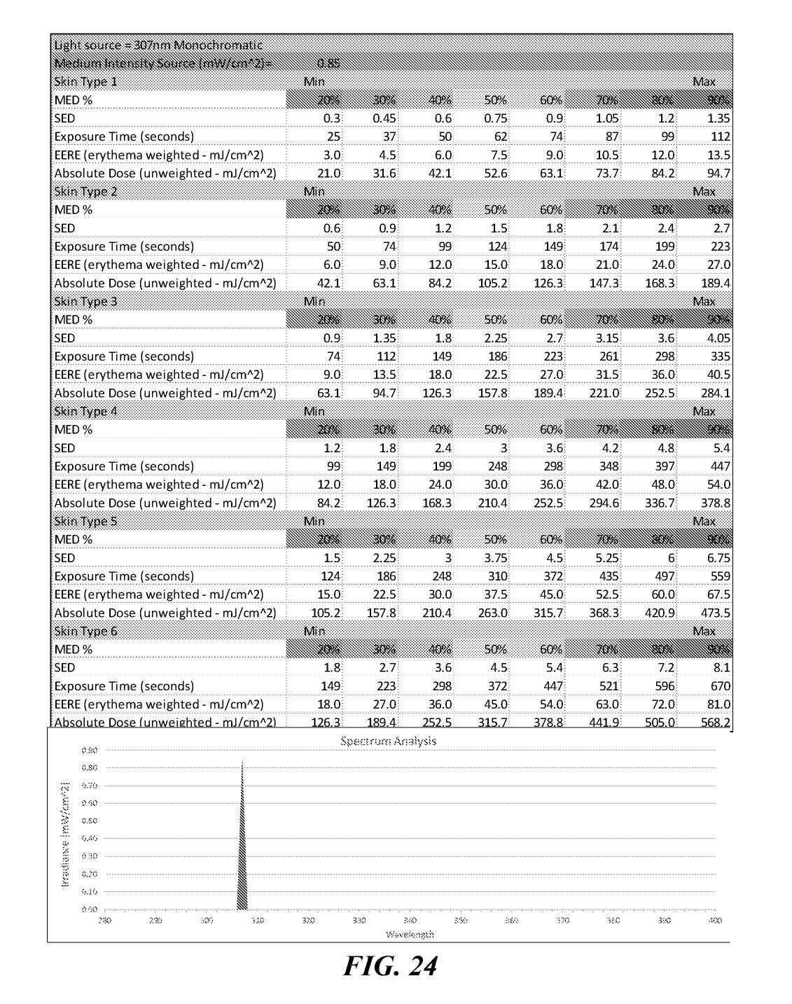

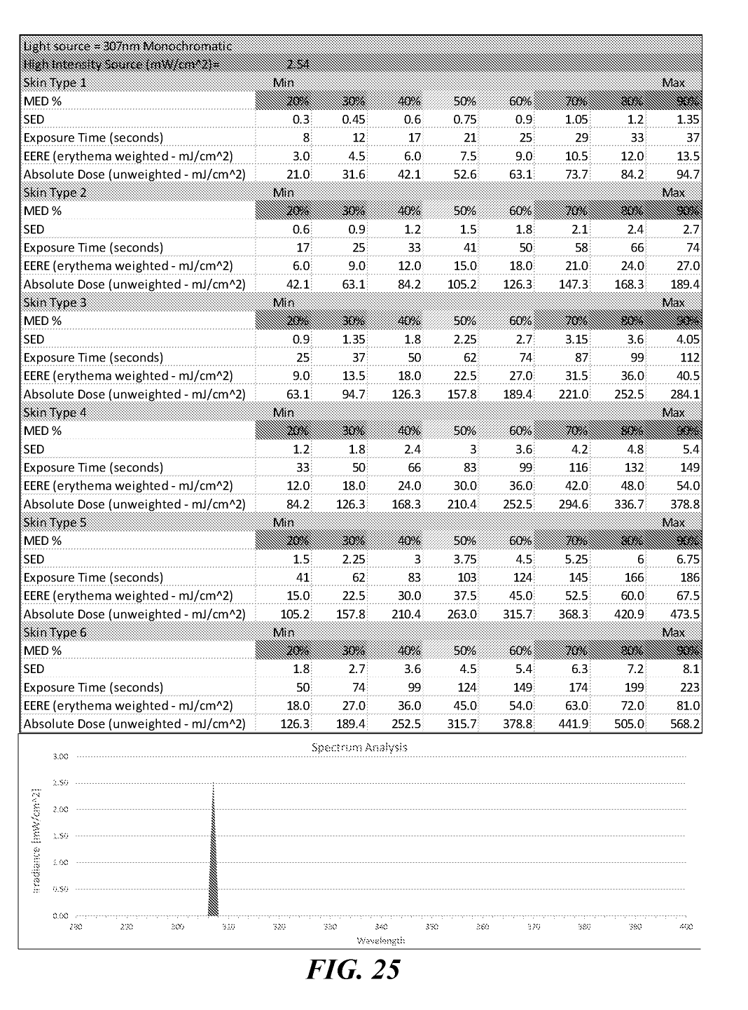

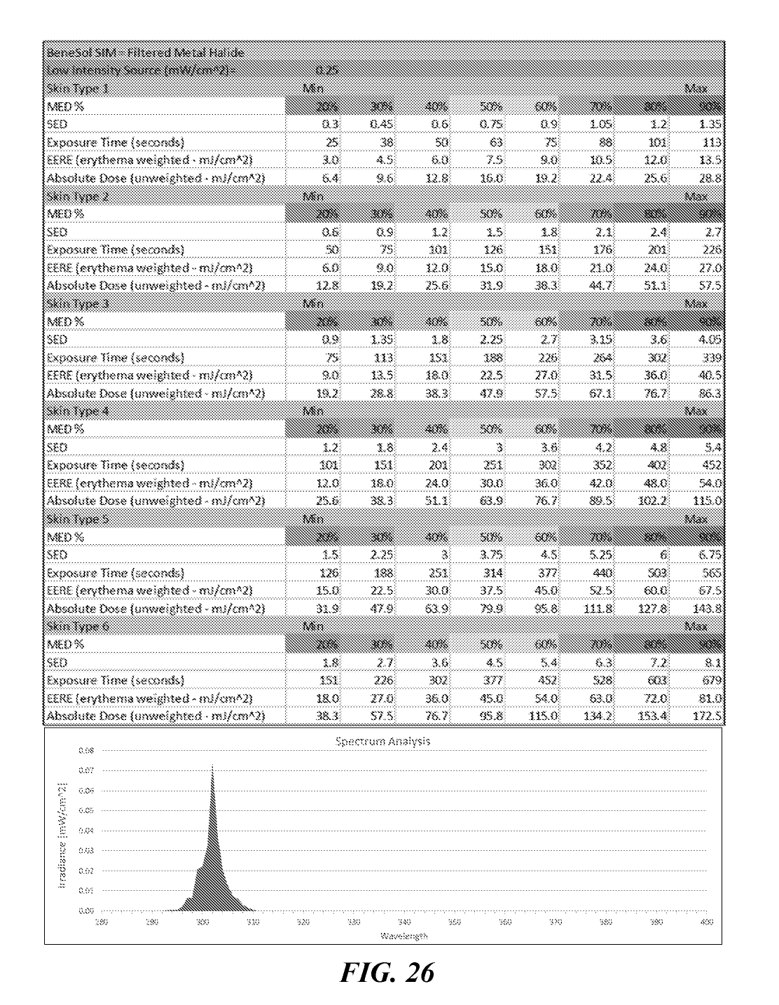

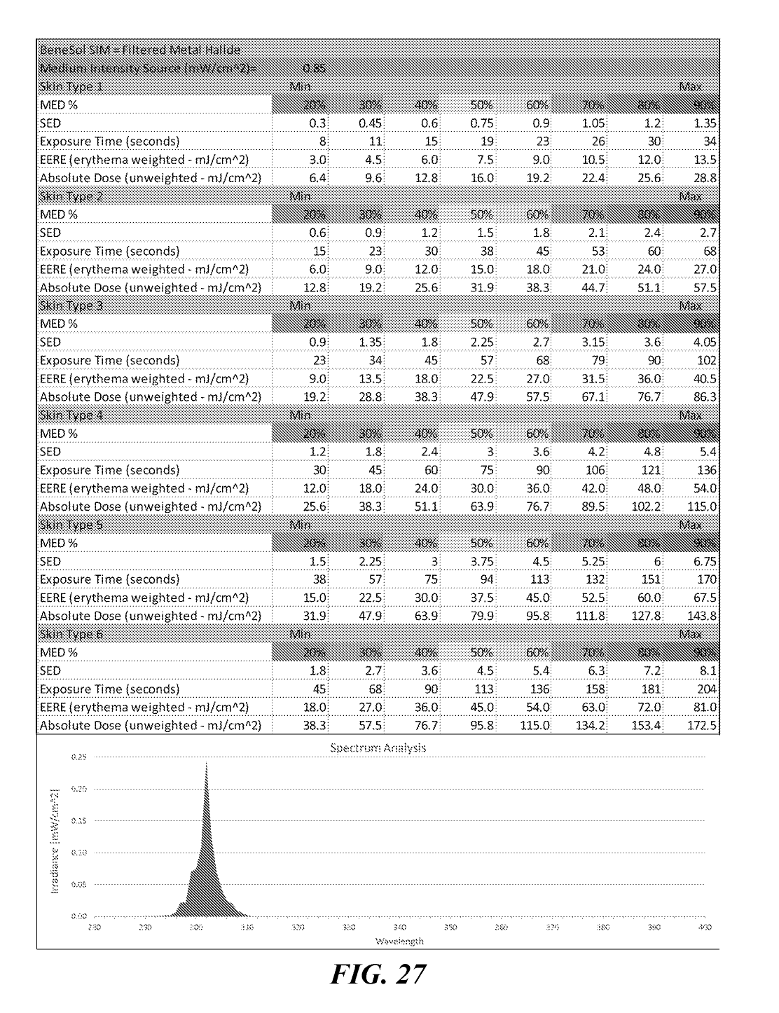

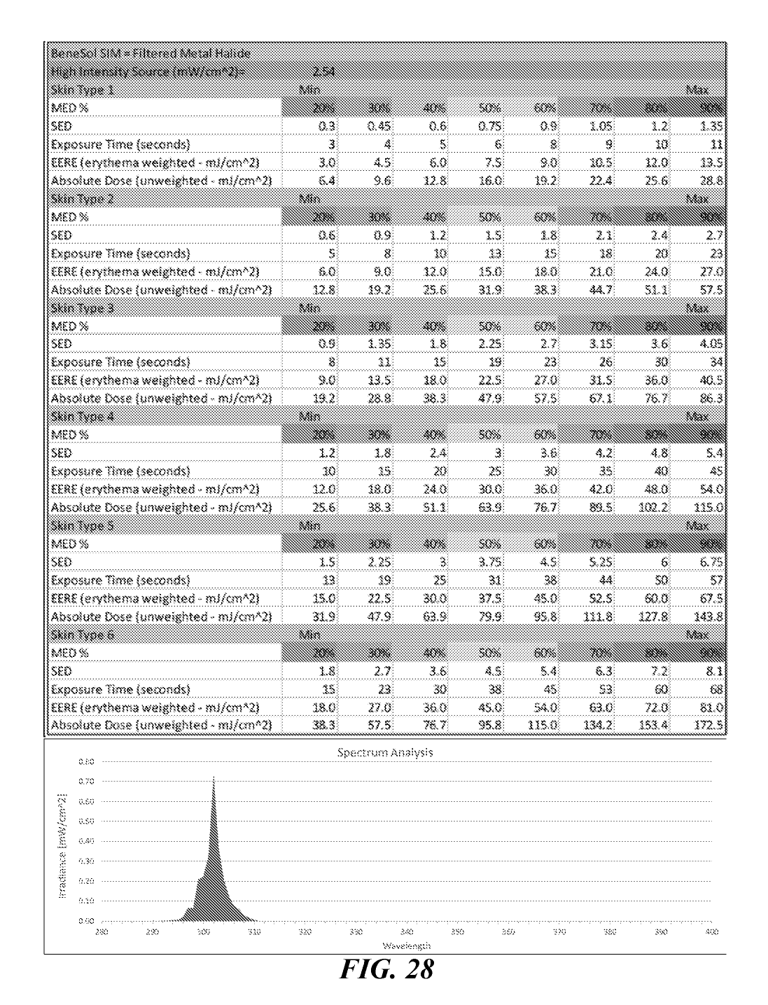

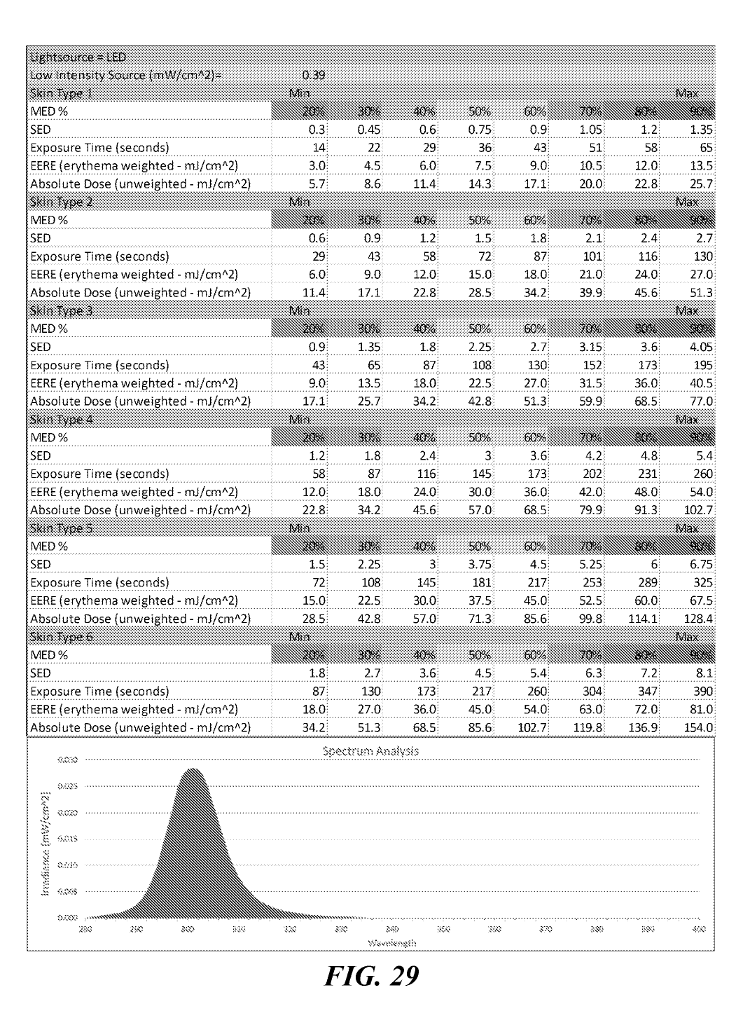

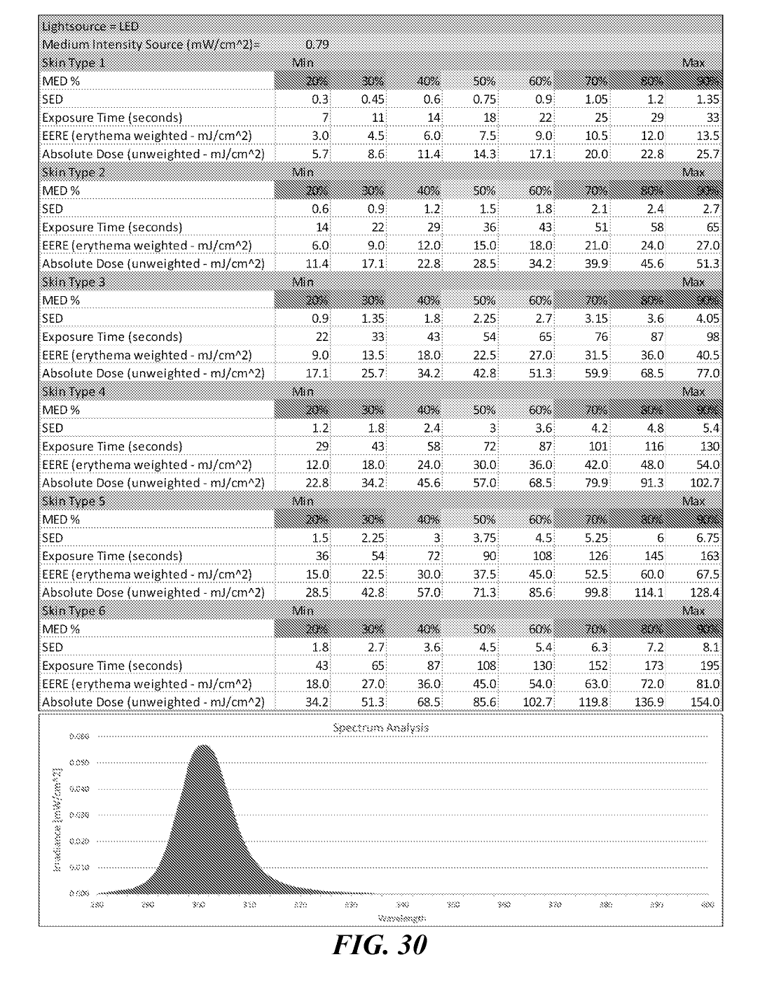

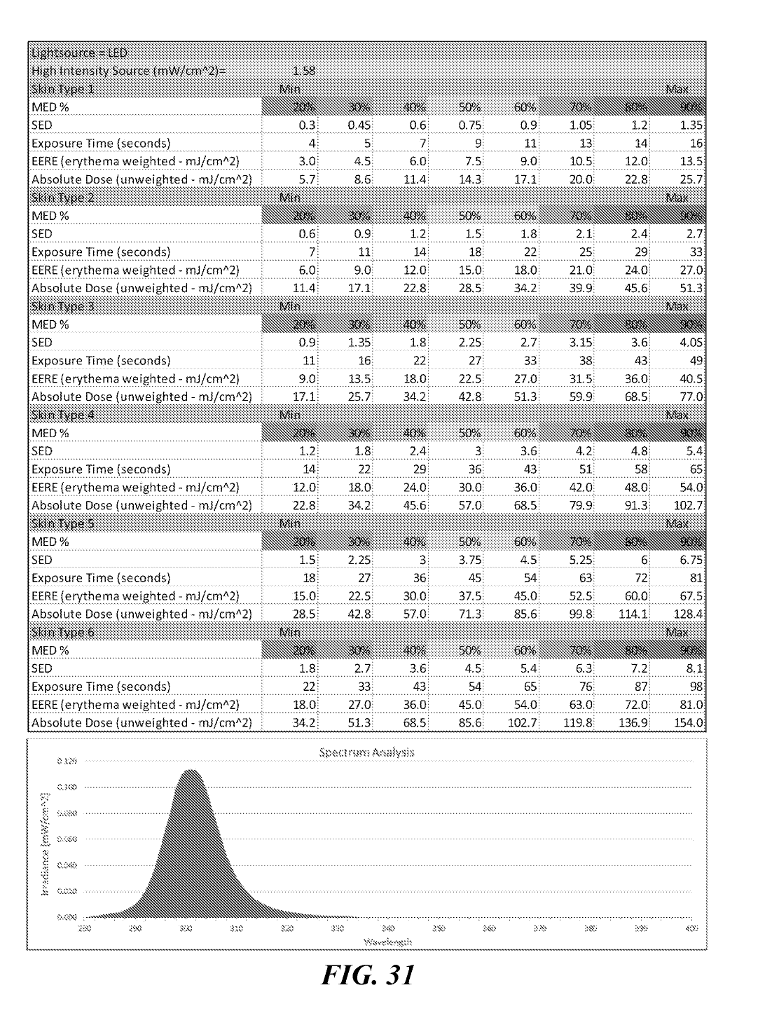

In practice, the parameters of phototherapy sessions for treating autoimmune disorders can be determined using dosage tables or charts for a selected phototherapy device with known or measured spectrum irradiance values and a selected MED dosage (e.g., 0.2 MED to 0.9 MED). For example, these dosage charts can be used to determine the SED, exposure time (e.g., seconds), absolute dose (mJ/cm₂), and EERE (mJ/cm²) for each Fitzpatrick skin type for the selected phototherapy device (given the spectrum irradiance measurement for that device). In certain embodiments, for example, a phototherapy device with focused UV range (e.g., 298 nm-307 nm) can have an MED dosage range of 0.2 MED (20% of 1 MED) to 0.9 MED (90% of I MED). Given this wavelength and MED dosage range, the calculation of exposure time, absolute dose (radiance density) and EERE can be calculated based on the device intensity and exact spectrum irradiance of the light source. This information can then be used to create a dosage chart showing the dosage range for each skin type for a specific phototherapy treatment device. FIGS. 17-31 illustrate such dosing tables for five phototherapy devices with different spectrum irradiances: a 298 nm monochromatic UV source (FIGS. 17-19), a 302 nm monochromatic UV source (FIGS. 20-22), a 307 nm monochromatic UV source (FIGS. 23-25), a 302 nm filtered metal halide UV source (FIGS. 26-28), and a 301 nm LED (FIGS. 29-31); and three different device intensity examples (i.e., low, medium, high) for each UV source. Using these dosage charts, a clinician can understand the range of operating parameters for a focused UV phototherapy device and select the desired parameters for a phototherapy session for a specific patient, varying the MED dosage accordingly. As shown in FIG. 19, for example, the 298 nm monochromatic high intensity UV source can deliver 0.2 MED to a patient having Skin Type 1 in a phototherapy session having a totally exposure time of just I second and an absolute irradiance of only 3.0 mJ/cm2. As shown in FIG. 23, using the 307 nm monochromatic low intensity UV source to deliver 0.9 MED to a patient having Skin Type 6 requires a phototherapy session of 37.25 minutes and has an absolute irradiance of 568.2 mJ/cm2.

### Selected Embodiments of Phototherapeutic Systems

FIG. 32 is an isometric view of a high-energy phototherapeutic apparatus or system (“system 3200”) for focused UV radiation configured in accordance with an embodiment of the present technology. The system 3200 includes a plurality of focused UV radiation fixtures or assemblies 3210 (“radiation assemblies 3210”) that emit energy within a predetermined wavelength range (e.g., about 298-307 nm, 298-304 nm, 300-305 nm, etc.), and limit or filter out a substantial portion of UV energy outside of the target wavelength range. For example, the system 3200 can be used to emit UVB radiation within the optimum wavelength range shown in the combination phototherapy action spectrum of FIG. 15. Each radiation assembly 3210 can emit energy having a substantially similar wavelength and similar intensity as the other radiation assemblies 3210 of the system 3200, or the emitted wavelengths and intensities of the individual radiation assemblies 3210 within the system 3200 may differ. In the illustrated embodiment, the radiation assemblies 3210 are carried by two housings, arms, or columns (identified individually as a first column 3230a and a second column 3230b, and referred to collectively as columns 3230) that are mounted on or otherwise attached to a pedestal or base 3232, and the radiation assemblies 3210 are directed generally inward toward a central portion 3234 of the base 3232. The base 3232 and the columns 3230 together define an irradiation zone in which a human can be exposed to focused UVB energy emitted by the radiation assemblies 3210. When a user (e.g., a human) stands on or is otherwise positioned at the central portion 3234 of the base 3232, the radiation assemblies 3210 can irradiate the user's skin to treat autoimmune disorders, stimulate vitamin D production in the skin, and/or treat other indications that may benefit from exposure to the predetermined wavelength range. In various embodiments, the central portion 3234 of the base 3232 and/or the columns 3230 may rotate relative to each other to expose all sides of the user's body to the energy emitted by the radiation assemblies 3210.

The system 3200 can provide an at least substantially uniform distribution of irradiation intensity by taking into account various features of the system 3200. For example, in the embodiment illustrated in FIG. 32 the radiation assemblies 3210 in the first column 3230a can be vertically offset from the radiation assemblies 3210 in the second column 3230b to prevent the irradiation from radiation assemblies 3210 of the first column 3230a from directly overlapping the irradiation from the radiation assemblies 3210 of the second column 3230b. For example, the radiation assemblies 3210 in the first column 3230a can be offset from radiation assemblies 3210 in the second column 3230b by about one radius of an individual radiation assembly 3210. This staggering of the radiation assemblies 3210 can provide a more uniform intensity of irradiation along the length of the columns 3230 and prevent certain areas of a user's skin from being exposed to more irradiation than others. In other embodiments, the system 3200 can include different features and/or other radiation assembly configurations to enhance the uniformity of the radiation emitted by the radiation assemblies 3210 and/or manipulate the direction in which the radiation is projected. For example, the radiation assemblies 3210 can include one or more lenses configured to diffuse or bend the light in a manner such that the light is evenly distributed across the irradiation zone or a portion thereof. In further embodiments, uniform emissions can be provided by an optical diffuser that diffuses, spreads out, or scatters light in a predetermined manner. For example, the lenses or diffusers can include ground glass diffusers, teflon diffusers, holographic diffusers, opal glass diffusers, and greyed glass diffusers. In still further embodiments, uniform emissions can be provided by selecting the distance the patient must be positioned away from the radiation assemblies 3210 to receive substantially uniform irradiation distribution, and/or the output of the system 3200 may be adjusted by changing the energy input, the number of lamps, lens specifications, and/or filtration parameters.

In further embodiments, the system 3200 can include columns 3230 with fewer than or more than the eight radiation assemblies 3210 shown in FIG. 32 (e.g., one radiation assembly, two radiation assemblies, four radiation assemblies, nine radiation assemblies, etc.), a single column 3230 of radiation assemblies 3210, more than two columns 3230 of radiation assemblies 3210 (e.g., four columns, six columns, etc.), and/or the radiation assemblies 3210 can be arranged in other suitable configurations. For example, the radiation assemblies 3210 can be carried by a housing that at least substantially encloses the irradiation zone and directs radiation inward toward an enclosed space defined by the housing.

The system 3200 can emit high intensity focused UVB radiation to provide therapeutic effects on autoimmune disorders or other indications, and/or facilitate vitamin D production in the skin during relatively short phototherapy sessions. For example, the apparatus 3200 can provide a sufficient amount of irradiation during a phototherapy session (e.g., 30 seconds, 1 minute, 2 minutes, 5 minutes, 10 minutes, etc.) to stimulate the production of a weekly or monthly dose of vitamin D. In various embodiments, the exposure time of each phototherapy session can be selected based on the on the user's skin type and/or the intensity of the radiation assemblies 3210. The user's skin type can be determined based on one or more mechanisms, such as one or more detectors that measures skin reflectance, color, and/or absorption and/or a questionnaire that is used to determine the user's Fitzpatrick skin type. More specifically, the user's skin type can also be determined by answering a series of questions related to the Fitzpatrick Skin Type scale (e.g., on an automated user interface of the system 3300), determined automatically using a sensor or detector on the housing of the system 330X) and/or operably coupled to the system 3300 that measures the reflectance, absorption, color, and/or other features related to skin type, and/or determined using a grid that allows the user or clinician to match the patient's skin tone to predetermined skin characteristics (e.g., fair, burns quickly, burns moderately; tans easily, etc.) and/or skin images of colors. In other embodiments, the patient's skin type can be determined automatically or manually using other suitable mechanisms and methods for determining skin type. Once the skin type of the user has been determined, the dosage of UVB emissions emitted by the system 3200 can be determined (e.g., via a controller). For example, the lighter the user's skin tone, the less exposure time necessary to obtain the desired level of UVB exposure in the user's skin or the less exposure time allowed to avoid overexposing the user's skin. As another example, the higher the intensity of the energy provided by the system 3200, the less exposure time necessary to obtain the desired irradiation for phototherapy. In certain embodiments, the amount of UVB emissions provided to each user can be selected using the dosage tables shown in FIGS. 17-31.

As shown in FIG. 32, each radiation assembly 3210 can include a UV radiation source 3212, a reflector 3236 partially surrounding the UV radiation source 3212, and a filter 3238 forward of the radiation source 3212. The radiation source 3212 can emit high energy (e.g., UV light), and at least some of the energy can contact the reflector 3236 (e.g., a mirrored substrate or coating) before exiting the radiation assembly 3210. The reflector 3236 can divert or otherwise direct the light forward toward the filter 3238 where light within a predetermined bandwidth (e.g., 6 nm, 8 nm, 16 nm, etc.) can exit the radiation assembly 3210. In certain embodiments, the reflector 3236 is curved around the radiation source 3212 such that the light emitted by the radiation source 3212 at least substantially collimates upon contact with the reflector 3236. The substantially collimated beam of light can then travel forward toward the filter 3238, and pass through the filter 3238 at the same or similar angle of incidence (e.g., a significant portion of the energy at about 0°, greater than 75% of the energy at less than 15°) to provide substantially uniform filtering of the light. In other embodiments, the radiation assemblies 3210 may not include the reflector 3236, and/or the radiation assemblies 3210 can include other features that at least substantially collimate the radiation emitted from the radiation sources 3212.

The radiation assemblies 3210 can further include one or more lenses 3233 positioned forward of (i.e., within the emission path of) the UV radiation sources 3212 to diffuse or otherwise manipulate the filtered light such that emissions from the radiation sources 3212 pass through the lenses 3233 before irradiating the human patient. For example, once the light is filtered via the filters 3238, the light can pass through the lenses 3233 to diffuse or otherwise spread the emitted light. In various embodiments, each radiation assembly 3210 can include one or more lenses 3233 positioned over the corresponding UV radiation source 3212, whereas in other embodiments a single lens 3233 can be positioned over plurality of radiation sources 3212. In certain embodiments, the filter 3238 can be integrated with the 3233. For example, the lens 3233 can include a first portion (e.g., a filtering element or portion) facing the UV radiation source 3212 that filters the emissions from the radiation source 3212 and a second portion (e.g., a lensing element or portion) spaced apart from the radiation source 3212 by the first portion that provides the diffusion or lensing of the filtered light. The filtering portion can be a substantially flat surface on which the filter 3238 (e.g., an interference coating) is disposed such that the light emitted by the UV radiation source 3212 (e.g., substantially collimated light) contacts the filter 3238 at substantially the same angle. The filtered energy can then move through lensing portion that diffuses, uniformly distributes, and/or otherwise shape the energy before it is emitted toward the user in the central portion 3234. In certain embodiments, the lens 3233 can be doped with a material to simultaneously act as an absorption filter and a lensing element. This absorption filter could remove broad ranges of light emitted by the UV radiation source 3212 and outside of the predetermined spectrum, such as infrared light, visible light, etc. Absorption filters generally absorb wide ranges of light, but have broad transition zones for filtering out light that prevent them from filtering out light within a small bandwidth (e.g., within a 10 nm range, a 20 nm range, a 100 nm range, etc.). Accordingly, in this embodiment, further filtering could be performed by a separate filter (e.g., via an interference coating on a substrate) to filter light outside of a predetermined spectrum. In other embodiments, the lens 3233 may be separate from the filter 3238 such that emissions from the UV radiation source 3212 first pass through the filter 3238 and then through the lens 3233.

The radiation source 3212 can include a metal halide lamp, which is a type of high-intensity discharge (“HID”) lamp that generates light by producing an electric arc through a gaseous mixture between two electrodes in an arc tube or envelope. The arc length (i.e., about the distance between the electrodes) of the metal halide lamp can be relatively small with respect to radiation assembly 3210 as a whole such that the metal halide lamp acts similar to a point source to facilitate collimation of the light. In other embodiments, the metal halide lamp can have larger or smaller arc lengths depending on the configuration of the metal halide lamp and the sizing of the other components of the radiation assembly 3210 (e.g., the reflector 3236). In other embodiments, the radiation source 3212 may include different types of high-energy UVB-emitting sources, such as mercury arc lamps, pulse and flash xenon lamps, halogen lamps, and fluorescent lamps.

When using metal halide lamps as the radiation source 3212, the gas mixture in the arc tube of the metal halide lamp can be selected to increase the UVB content of the emissions of the metal halide lamp. For example, the gas mixture can be doped to generate about 6% of the total emissions in the UVB range (e.g., about 280-315 nm) in comparison to normal tanning bed lamps that have about 1% of their emissions in the UVB range. The increased UVB content of the emissions can increase the intensity of the UVB emitted by the radiation assembly 3210, and therefore may decrease the overall exposure time necessary to achieve a desired phototherapy. Based on test data, it is believed that large portions of the emissions of doped metal halide lamps have wavelengths of about 300-305 nm. As discussed above with respect to FIG. 15, the combination phototherapy action spectrum suggests that an optimal wavelength range for treatment of autoimmune disorders is about 298-307 nm. Accordingly, metal halide lamps are uniquely suited for promoting vitamin D production in the skin and immune responses for autoimmune disorders, and may require less filtering than other types of UV radiation sources.

The filter 3238 can be a narrow pass filter that prevents UVB radiation outside of a predetermined bandwidth from exiting the radiation assembly 3210. In certain embodiments, the filter 3238 can include a substrate (e.g., glass, plastic, etc.) and at least one interference coating applied to the substrate. The coating can be sprayed onto the substrate and/or otherwise disposed on the substrate using methods known to those skilled in the art. Substrates and interference coatings that provide at least some filtering of UV radiation outside of a predetermined spectrum are available from Schott of Elmsford, N.Y. In various embodiments, other portions of the radiation assemblies 3210 can include interference coatings and/or other filtering features that block at least some radiation outside of the desired wavelength spectrum. For example, an absorption filter can be incorporated into the envelope of a metal halide lamp or the substrate of the filter 3238 (e.g., metal additives can be incorporated into the quartz of the lamp and/or filter substrate). The combination phototherapy action spectrum described above with reference to FIG. 15 can be used to determine the most efficient wavelength for phototherapy, and a narrow pass filter can be designed or selected to emit radiation centered at the predetermined wavelength. For example, in certain embodiments, the filter 3238 (by itself or in combination with an absorption filter) can at least substantially block UVA, UVB, and UVC radiation outside of a predetermined spectrum (e.g., about 298-307 nm). In other embodiments, the filter 3238 can at least substantially block UVB radiation outside of different bandwidths (e.g., a 4 nm spectrum, a 6 nm spectrum, an 8 nm spectrum, a 12 nm spectrum, a 16 nm spectrum, etc.), and/or the spectrum can be centered around other suitable wavelengths for treating autoimmune disorders and/or producing vitamin D (e.g., 298 nm, 300 nm, 302 nm, etc.). The concentrated UVB radiation provided by the system 3200 can deliver a large amount of UVB radiation within the desired wavelength range (e.g., shown in FIG. 15) within a relatively short phototherapy session (e.g., less than 15 minutes, less than 10 minutes, less than 5 minutes, less than 2 minutes, less than 1 minute, etc.). The UVB radiation can be distributed in a substantially uniform emission pattern such that the exposed area of the user's skin (i.e., the treatment area) is exposed to a substantially uniform intensity of light. The dosage provided to each user can be selected based on the dosage tables described above with respect to FIGS. 17-31.

FIG. 33 is an isometric view of a low-energy phototherapeutic apparatus or system (“system 3300”) for focused UV radiation configured in accordance with another embodiment of the present technology. The system 3300 can include a wearable substrate 3310 and a plurality of low-intensity radiation sources 3320 (e.g., 3 Watts or less), such as a plurality of LEDs. As used herein, a wearable substrate refers to an article or apparatus that can come in close proximity to a patient's skin (e.g., within 3 cm of the patient's skin) such that the patient comes in close proximity to the radiation sources 3320. In the embodiment illustrated in FIG. 33, for example, the wearable substrate 3310 is a blanket or pad that a patient can lay on top of or under. In other embodiments, the wearable substrate 3310 may be other items, such as bands that wrap around portions of a patient's body (e.g., a patient's leg, arm, torso, wrist, etc.), sleeves, clothing (e.g., tightly fitting shirts or pants), and/or other articles that can carry the low-intensity radiation sources and can be held in close proximity to the patient's skin. The wearable phototherapy system 3300 can provide a substantially uniform and constant level of radiation intensity across the portion of the wearable substrate 3310 including the radiation sources 3320. This allows phototherapy to be delivered to selective and scalable treatment areas.

The radiation sources 3320 of the system 3300 can be arranged on the wearable substrate 3310 in tightly packed arrays. In various embodiments, the radiation sources 3320 are spread evenly across the wearable substrate 3310 (e.g., as shown in FIG. 33), whereas in other embodiments the radiation sources 3320 are spaced in specific sections or unevenly distributed across the wearable substrate 3310. The radiation sources 3320 can be LEDs that emit light with relatively monochromatic wavelength emissions (e.g., 298 nm, 300 nm, 302 nm, 305 nm, etc.) or at a plurality of different wavelengths within a predetermined narrow bandwidth (e.g., 10 nm bandwidth, 7 nm bandwidth, 5 nm bandwidth, etc.) suitable for treating dermatological disorders, vitamin D deficiency, autoimmune disorders, and/or other indications. For example, the wavelengths of the LEDs can be selected using the methods and action spectra described above with respect to FIGS. 1-15. In certain embodiments, the LEDs can emit wavelengths between 298 nm and 307 nm. In other embodiments, the LEDs can have one or more different wavelengths, such as wavelengths ranging from 295 nm to 310 nm or therebetween. The monochromatic output of the LEDs may reduce or eliminate the amount of filtering necessary to provide UVB radiation within a predetermined spectrum. Suitable LEDs are available from, for example, Sensor Electronic Technology, Inc. of Columbus, S.C.

The individual radiation sources 3320 can also include one or more lenses 3330 (identified individually as a first lens 3330a and a second lens 3330b). Individual lenses, such as the first lens 3330a, can be positioned over each individual radiation source 3320. In other embodiments, a larger lens, such as the second lens 3330b, can extend over two or more of the radiation sources 3320 (e.g., all of the radiation sources 3320 on the wearable substrate 3310). In certain embodiments, the larger second lens 3330b can be used in conjunction with the individual first lenses 3330a. The lenses 3330 can manipulate the emissions from the radiation sources 3320 to diffuse, spread, or otherwise change the emission pattern of the radiation sources 3320. In further embodiments, the system 3300 can include other features that diffuse or spread the emitted light at least substantially evenly across a portion of the wearable substrate 3310 or the entire surface area of the wearable substrate 3310.

The intensity of the array of radiation sources 3320 can be selected by adjusting various parameters of the radiation sources 3320 and the array of the radiation sources 3320. For example, the intensity of the radiation source array can be increased by increasing the input energy delivered to the radiation sources 3320 (e.g., by changing the power source or controls thereon), increasing the quantity of radiation sources 3320 per unit area, decreasing the distance between the radiation sources and the treatment area on the patient (e.g., 0-3 cm, within 4 cm, within 5 cm, etc.), decreasing the degree of light spreading of the lens(es) 3330 on the radiation sources 3320, and/or changing other features of the radiation source array that impact the radiation intensity. Conversely, the intensity of the radiation source array can be decreased by decreasing the level of energy delivered to the radiation sources 3320, decreasing the quantity of radiation sources 3320 per unit area, increasing the distance between the radiation sources 3320 and the treatment area on the patient, increasing the degree of light spreading of the lens(es) 3330, and/or changing other features of the radiation source array that impact the radiation intensity.

As shown in FIG. 33, the system 3300 can further include a controller 3350 operably coupled to the radiation sources 3320 on the wearable substrate 3310. The controller 3350 can be coupled to radiation sources 3320 via a wired connection line 3360 (e.g., an electrical cord) or via a wireless connection (e.g., Bluetooth, internet, intranet, etc.). The controller 3350 can be manipulated by an operator (e.g., a clinician, a technician, and/or the user) to activate and deactivate the system 3300, as well as adjust various parameters of the system 3300. These parameters can include, for example, the level of energy delivered to the radiation sources 3320. As described in further detail below, the controller 3350 can include various automated programs and algorithms that adjust the parameters of the system 3300. For example, the controller 3350 can adjust the dosage provided by the system 3300 using the dosage tables described above with respect to FIGS. 17-31.

In operation, the system 3300 can provide an at least substantially uniform distribution of irradiation intensity by taking into account various features of the system, such as the distance between the radiation sources 3320 and the treatment site on the patient, the spacing of the radiation sources 3320 with respect to each other, and/or the shape of the lenses 3330 on the radiation sources 3320. For example, the radiation source array can be arranged such that at least a major portion of emission patters of the individual radiation sources 3320 do not overlap each other. The lenses 3330 on the radiation sources 3320 can be used to expand or contract the emissions of the individual radiation sources 3320 such that they do not overlap each other. In certain embodiments, radiation sources 3320 are spaced apart by a distance that avoids any overlapping emissions, and therefore leaves some portions of the treatment area (e.g., the area of skin facing the wearable substrate 3310) unexposed from the emissions.

In various embodiments, the system 3300 can be configured such that the radiation sources 3320 remain at a constant distance from the treatment area during the phototherapy session to maintain the uniform exposure to the radiation sources 3320. Accordingly, the wearable substrate 3310 can be placed in direct contact with the treatment area. In certain embodiments, the system 3300 can include a sensor 3340 that indicates when the radiation sources 3320 are appropriately placed on the skin to confirm direct skin contact before and/or during operation of the system 3300 during a phototherapy session. The embodiment illustrated in FIG. 33 includes a single sensor 3300. However, in other embodiments, the system 3300 can include a plurality of sensor 3340 spaced across the wearable substrate to confirm contact with the patient's skin.

In further embodiments, the sensor 3340 can include a detector that measures skin reflectance and/or color to automatically determine a patient's skin type before the phototherapy is applied. In other embodiments, the sensor 3340 can measure other characteristics related to skin type. As described above, this information can be used in determining the correct dosage to provide to the patient (e.g., as shown in reference to FIGS. 17-31). The controller 3350 can then be used to adjust the parameters of the system 3300, such as phototherapy duration and energy input, in response to the measured skin type. In other embodiments, this information can be manually entered into the controller 3350. In further embodiments, skin type can be determined by answering questions a series of questions related to the Fitzpatrick Skin Type scale (e.g., on an automated user interface of the system 3300), using a grid that allows the user or clinician to match the patient's skin tone to predetermined skin characteristics (e.g., fair, burns quickly, burns moderately; tans easily, etc.) and/or skin images of colors, and/or using other suitable mechanisms and methods for determining skin type.

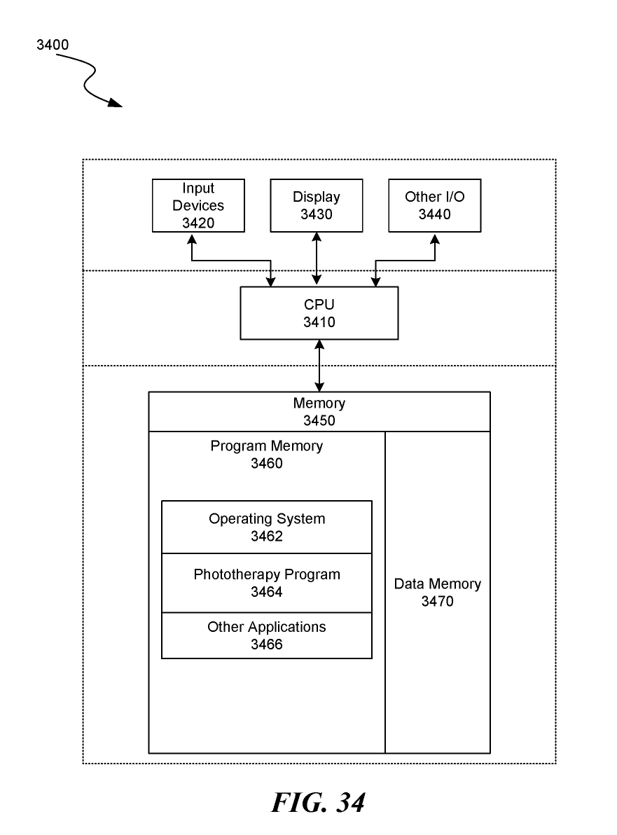

FIG. 34 is a block diagram illustrating an overview of devices on which some implementations of the disclosed technology can operate. The devices can comprise hardware components of a device 3400 for selecting parameters for phototherapy sessions that may affect phototherapy dosage. This device 3400 may be a controller, such as the controller 3450 of FIG. 34, that operates a phototherapy system (e.g., the phototherapy systems 3200 and 3300 described above with reference to FIGS. 32 and 33). Device 3400 can include, for example, one or more input devices 3420 providing input to a central processing unit (“CPU”; processor) 3410, notifying the CPU 3410 of actions. The actions are typically mediated by a hardware controller that interprets the signals received from the input device and communicates the information to the CPU 3410 using a communication protocol. The input devices 3420 include, for example, a receiver for receiving signals from sensors (e.g., skin contact sensors, distance sensors, skin irradiance detectors, other skin type sensors, etc.), a mouse, a keyboard, a touchscreen, an infrared sensor, a touchpad, a wearable input device, a camera- or image-based input device, a microphone, and/or other user input devices.

The CPU 3410 can be a single processing unit or multiple processing units in a device or distributed across multiple devices. CPU 3410 can be coupled to other hardware devices, for example, with the use of a bus, such as a PCI bus or SCSI bus. The CPU 3410 can communicate with a hardware controller for devices, such as for a display 3430. The display 3430 can be used to display text and graphics. In some examples, the display 3430 provides graphical and textual visual feedback to a user, such as the parameters of a phototherapy session, a summary of indices detected by a detector coupled to the device 3400, and/or other suitable information. In some implementations, the display 3430 includes the input device as part of the display, such as when the input device is a touchscreen or is equipped with an eye direction monitoring system. In some implementations, the display 3430 is separate from the input device 3420. Examples of display devices are: an LCD display screen, an LED display screen, a projected, holographic, or augmented reality display (such as a heads-up display device or a head-mounted device), and so on. Other l/O devices 3440 can also be coupled to the processor, such as a network card, video card, audio card, USB, firewire or other external device, camera, printer, speakers, CD-ROM drive, DVD drive, disk drive, or Blu-Ray device.

In some implementations, the device 3400 also includes a communication device capable of communicating wirelessly or wire-based with a network node. The communication device can communicate with another device or a server through a network using, for example, TCP/IP protocols. Device 3400 can utilize the communication device to distribute operations across multiple network devices.

The CPU 3410 can have access to a memory 3450. A memory includes one or more of various hardware devices for volatile and non-volatile storage, and can include both read-only and writable memory. For example, a memory 3450 can include random access memory (RAM), CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. The memory 3450 can include program memory 3460 for storing programs and software, such as an operating system 3462, a phototherapy program 3464, and other application programs 3466. The phototherapy program 3464, for example, can include one or more algorithms for determining the parameters of a phototherapy system (e.g., the system 3200 and 3300 described in FIGS. 32 and 33) to provide proper dosage for a patient, analyzing parameters of a system during a phototherapy session, and/or providing a recommendation for a specific therapy or specific parameters of a therapy that a clinician or other user can then adjust. The memory 3450 can also include data memory 970 including recorded data from a cardiac detector, patient data, patient skin types, algorithms related to phototherapy analysis, configuration data, settings, user options or preferences, etc., which can be provided to the program memory 3460 or any element of the device 3400. For example, the data memory 3470 can store each patient's skin type, previous phototherapy session data, and/or other information, and the phototherapy program 3464 can recall this information during the patient's next phototherapy session to determine phototherapy parameters that provide the correct dosage for the patient.

Some implementations can be operational with numerous other general purpose or special purpose computing system environments or configurations. Examples of well-known computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, set-top boxes, programmable consumer electronics, network PCs, minicomputers, mainframe computers, distributed computing environments that include any of the above systems or devices, or the like.

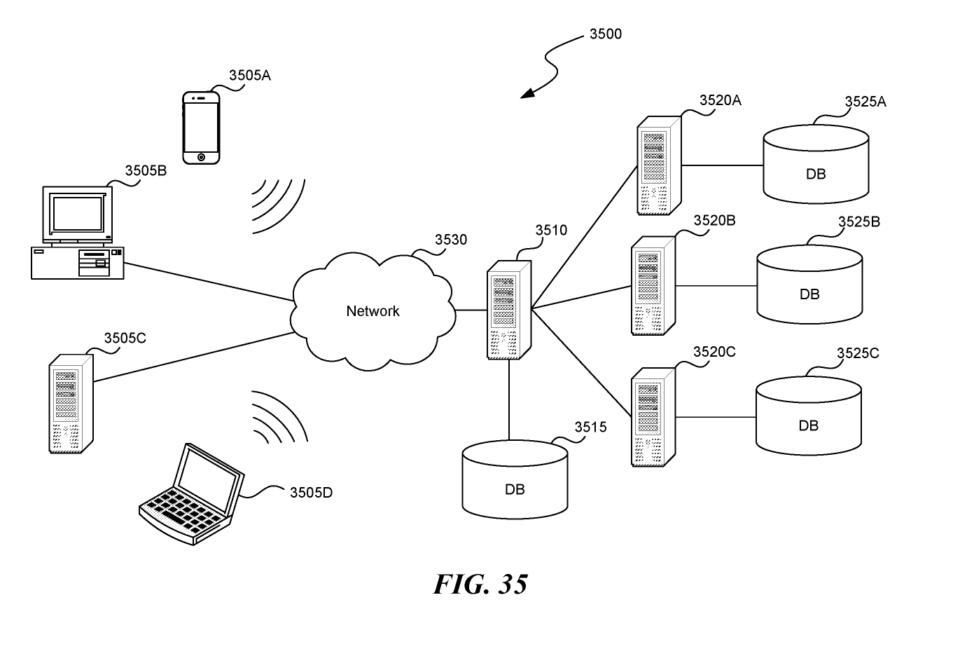

FIG. 35 is a block diagram illustrating an overview of an environment 35000 in which some implementations of the disclosed technology can operate. The environment 35000 can include one or more client computing devices 3505A-D (identified collectively as “client computing devices 3505”), examples of which can include the device 3400 of FIG. 34. The client computing devices 3505 can operate in a networked environment using logical connections through a network 3530 to one or more remote computers, such as a server computing device 3510.

In some implementations, server 3510 can be an edge server that receives client requests and coordinates fulfillment of those requests through other servers, such as servers 3520A-C. The server computing devices 3510 and 3520 can comprise computing systems, such as device 3400 (FIG. 34). Though each server computing device 3510 and 3520 is displayed logically as a single server, the server computing devices 3510 and 3520 can each be a distributed computing environment encompassing multiple computing devices located at the same or at geographically disparate physical locations. In some implementations, each server 3520 corresponds to a group of servers.

The client computing devices 3505 and the server computing devices 3510 and 3520 can each act as a server or client to other server/client devices. The server 3510 can connect to a database 3515. The servers 3520A-C can each connect to a corresponding databases 3525A-C. As discussed above, each server 3520 can correspond to a group of servers, and each of these servers can share a database or can have their own database. The databases 3515 and 3525 can warehouse (e.g. store) information such as algorithms for deriving phototherapy parameters for specific dosages and specific phototherapy system, patient information, and/or other information necessary for the implementation of the systems and methods described above with respect to FIGS. 1-34. Though the databases 3515 and 3525 are displayed logically as single units, the databases 3515 and 3525 can each be a distributed computing environment encompassing multiple computing devices, can be located within their corresponding server, or can be located at the same or at geographically disparate physical locations.

The network 3530 can be a local area network (LAN) or a wide area network (WAN), but can also be other wired or wireless networks. The network 3530 may be the Internet or some other public or private network. The client computing devices 3505 can be connected to the network 3530 through a network interface, such as by wired or wireless communication. While the connections between the server 3510 and servers 3520 are shown as separate connections, these connections can be any kind of local, wide area, wired, or wireless network, including the network 3530 or a separate public or private network.

### Phototherapy for Autoimmune Disorders

Ultraviolet phototherapy has been used for several years as a treatment for dermatological disorders because of the immune modulating response from the skin. Low serum 25-hydroxyvitamin D₃ is correlated to several autoimmune disorders, so increased blood concentration from UVB phototherapy may benefit those conditions. Calcitriol mediates an anti-inflammatory immune response and enhances regulatory T cell functionality. Dermal production of calcitriol through UVB phototherapy is expected benefit several inflammatory autoimmune conditions. Phototherapy using UVB can instantiate a favorable systemic immune response that produces photoproducts (ACTH, MSH and BE) shown to benefit several autoimmune conditions. A targeted UVB phototherapy device that maximizes immune response, calcitriol production and vitamin D₃ production is expected have multiple biological mechanisms of benefit for autoimmune conditions.

UV exposure-mediated immune response, calcitriol production, vitamin D₃ production and erythema are all highly wavelength dependent. In various embodiments, dosage for UV phototherapy is based on minimal erythemal dose (MED), which is dictated by the erythema action spectrum. Isolating and delivering to the skin a small wavelength range (e.g., 10 nm or less) of UV radiation focused between about 298 nm and 307 nm while minimizing or eliminating UV radiation outside this target range is expected maximize phototherapy efficacy for autoimmune disorders while minimizing or reducing total UV exposure.

There are numerous advantages associated with this a new phototherapy of isolating and delivering UV radiation focused around 302 nm for phototherapy. For example phototherapy treatments using the dosages and parameters outlined above can enhance the maximum efficacy of treatment of autoimmune disorders (e.g., MS), while also minimizing the exposure time and total UV exposure per phototherapy treatment of the autoimmune diseases based on the erythema action spectrum. The dosages and parameters can also be used to decrease or minimize the UV exposure per phototherapy treatment to achieve systemic immune suppression and biological response based on several immune response action spectra. In addition, the dosages and parameters can provide phototherapy treatments with reduced or minimized levels of UV exposure per phototherapy treatment session needed to successfully treat autoimmune disorders based on UV production of ACTH, MSH, and BE, autoimmune disorders based on the cutaneous production of vitamin D; and consequential correction of 25-hydroxyvitamin D₃ insufficiency, and/or autoimmune disorders based on the calcitriol action spectrum and resultant epidermal production of calcitriol. Moreover, the dosages and parameters can provide phototherapy treatment with reduced or minimized levels of UV exposure per phototherapy treatment session needed to achieve maximum cutaneous calcitriol production. Thus, the present disclosure provides systems and methods for an endogenous alternative for synthetic ACTH therapy used for MS and arthritis treatment and/or an endogenous alternative to relieve inflammatory pain related to many autoimmune conditions based on maximum dermal beta endorphin production.

### Examples

The following Examples are illustrative of several embodiments of the present technology.

1. A phototherapeutic system for treating an autoimmune disorders, the phototherapeutic system comprising:
a radiation source configured to emit light and having an intensity, wherein at least 75% of the light emitted by the radiation source has a target wavelength range with a bandwidth between 298 nm and 307 nm; and
a controller operably connected to the radiation source and configured to determine a dosage for a phototherapy session, wherein the dosage is equivalent to a product of the intensity of the radiation source and an exposure time of the radiation source, wherein the dosage has an upper bound less than 1 minimal erythema dose (MED), and wherein delivery of the dosage provides an immune response to treat the autoimmune disorder.

2. The phototherapeutic system of example 1 wherein the radiation source is configured to filter out a substantial portion of UV energy outside of the target wavelength range.

3. The phototherapeutic system of example 1 or 2 wherein the radiation source is configured to expose at least 30% of a patient's skin to the light emitted by the radiation source.

4. The phototherapeutic system of any one of examples 1-3 wherein the radiation source is a low-energy radiation source and is configured to be positioned within 3 cm of a treatment area

5. The phototherapeutic system of example 4 wherein the radiation source comprises an array of LEDs.

6. The phototherapeutic device of example 1, further comprising:
a wearable substrate, and
wherein the radiation source comprises a plurality of LEDs arranged on the wearable substrate and configured to emit light within a treatment area.

7. The phototherapeutic device of example 6 wherein the LEDs are configured to emit a substantially uniform UV radiation across the treatment area.

8. The phototherapeutic device of example 6 or 7, further comprising a sensor on the wearable substrate, wherein the sensor is configured to determine proximity of the radiation sources to a patient's skin.

9. The phototherapeutic device of any one of examples 1-6, further comprising a sensor configured to measure skin absorption, color, and/or reflection, wherein the controller is configured to select dosage based on the skin absorption, color, and/or reflection measured by the sensor.

10. The phototherapeutic device of example 1 wherein the radiation source comprises a plurality of high-energy radiation sources configured to emit light of substantially equal intensity to the treatment area

11. The phototherapeutic device of example 10 wherein the plurality of high-energy radiation sources are configured to be spaced apart from the treatment area by about 10-200 cm, and wherein variations in distances between the high-energy radiation sources and the treatment area are less than 50 cm.

12. The phototherapeutic system of example 1 wherein the radiation source comprises at least one of a narrow-band UVB source or a broad-band UVB source.

13. The phototherapeutic system of any one of examples 1-12 wherein the dosage of the radiation source is configured to produce at least one of Adrenocorticotropic Hormone (ACTH), Melanocyte Stimulating Hormone (MSH), or Beta Endorphin (BE).

14. The phototherapeutic system of any one of examples 1-13 wherein the dosage of the radiation source is configured to produce at least one of cis-urocanic acid or DNA pyrimidine dimers.

15. The phototherapeutic system of any one of examples 1-14 wherein the intensity of the radiation source is an erythemally weighted irradiance equal to a summation of the product of an absolute measured intensity for each wavelength of light emitted by the radiation source and an erythema reference action spectrum weighting factor.

16. The phototherapeutic system of any one of examples 1-15 wherein the radiation source comprises:
a UV radiation source and configured to emit energy;
a filter forward of the UV radiation source and configured to remove energy outside of the target wavelength range; and
a lens forward of the filter and configured to diffuse energy in a substantially uniform manner.

17. The phototherapeutic system of any one of examples 1-15 wherein the radiation source comprises:
a UV radiation source; and
a lens forward of the UV radiation source, wherein the lens includes a filtering portion facing the UV radiation source and configured to remove light outside of the target wavelength range and lensing element spaced apart from the UV radiation source by the filtering portion and configured to diffuse filtered light in a substantially uniform manner.

18. A phototherapeutic system for treating an autoimmune disorders, the phototherapeutic system comprising:
a radiation source configured to emit light and having an intensity, wherein at least 75% of the light emitted by the radiation source has a target wavelength range with a bandwidth between 298 nm and 307 nm; and
a controller operably connected to the radiation source and configured to determine a dosage for a phototherapy session, wherein the dosage is equivalent to a product of the intensity of the radiation source and an exposure time of the radiation source, wherein the dosage has an upper bound less than 10 standard erythema dose (SED), and wherein delivery of the dosage provides an immune response to treat the autoimmune disorder.

19. A method of treating autoimmune disorders with a phototherapy system, the method comprising:
determining a skin type of a user;
determining, via a controller, a dosage of phototherapy to deliver to the user during a phototherapy session, wherein the dosage is equivalent to a product of the intensity of a radiation source of a radiation assembly of the phototherapy device and an exposure time of the radiation source, and wherein the dosage has an upper bound less than 1 minimal erythema dose (MED); and
delivering the dose of phototherapy to a treatment area on the user via the phototherapy device, wherein delivering the dose of phototherapy comprises emitting light from the radiation assembly having one or more target wavelength ranges within a bandwidth of 298-307 nm, wherein delivery of the dose of phototherapy provides an immune response to treat the autoimmune disorder.

20. The method of example 19 wherein delivering the dose of phototherapy produces at least one of Adrenocorticotropic Hormone (ACTH), Melanocyte Stimulating Hormone (MSH), or Beta Endorphin (BE).

21. The method of example 19 or 20 wherein delivering the dose of phototherapy produces at least one of cis-urocanic acid or DNA pyrimidine dimers.

22. The method of any one of examples 1921 wherein determining the skin type of the user comprises measuring, via a sensor, skin reflectance, color, or absorption of the user.

23. The method of any one of examples 19-22, further comprising determining the intensity of the radiation source by summing the product of an absolute measured intensity for each wavelength of light emitted by the radiation source and an erythema reference action spectrum weighting factor.

24. The method of any one of examples 19-23 wherein:
delivering the dose of phototherapy comprises emitting light from a plurality of high-energy radiation sources; and
the method further comprises positioning the treatment area of the user apart from the radiation sources by less than 200 cm, wherein variations in distance between the high-energy radiation sources and the treatment area are less than 50 cm.

25. The method of any one of examples 19-24 wherein delivering the dose of phototherapy comprises delivering the dose of phototherapy to at least 30% of the user's skin.

26. The method of any one of examples 19-25 wherein:
delivering the dose of phototherapy comprises emitting light from a plurality of low-energy radiation sources arranged on a wearable substrate; and
the method further comprises positioning the treatment area of the user apart from the low-intensity radiation sources by less than 3 cm and maintaining a substantially uniform distance between the treatment area and the radiation sources during the exposure time.

27. The method of any one of examples 19-26, further comprising adjusting, via the controller, exposure time and intensity of the radiation source in relation to each other to select the dosage.

28. The method of any one of examples 19-27, further comprising filtering out a substantial portion of UV energy outside of the target wavelength range.

29. The method of any one of examples 19-28 wherein determining dosage of phototherapy comprises delivering the dosage of phototherapy based on the skin type of the user.

30. The method of any one of examples 19-29, further comprising:
storing the skin type of the user on a database remote from the phototherapy device; and
accessing the skin type of the user during subsequent phototherapy sessions to determine the dosage of phototherapy.

31. The method of any one of claims 19-30 wherein delivering the dose of phototherapy comprises:
filtering the light emitted from the radiation source to remove light outside of the target wavelength ranges; and
diffusing the filtered light with a lens to distribute the filtered light in a substantially uniform manner.

### CONCLUSION

From the foregoing, it will be appreciated that specific embodiments of the technology have been described herein for purposes of illustration, but that various modifications may be made without deviating from the disclosure. Certain aspects of the new technology described in the context of particular embodiments may be combined or eliminated in other embodiments. Additionally, although advantages associated with certain embodiments of the new technology have been described in the context of those embodiments, other embodiments may also exhibit such advantages and not all embodiments need necessarily exhibit such advantages to fall within the scope of the technology. Accordingly, the disclosure and associated technology can encompass other embodiments not expressly shown or described herein.

I/We claim:

1. A phototherapeutic system for treating an autoimmune disorders, the phototherapeutic system comprising:
a radiation source configured to emit light and having an intensity, wherein at least 75% of the light emitted by the radiation source has a target wavelength range with a bandwidth between 298 nm and 307 nm; and
a controller operably connected to the radiation source and configured to determine a dosage for a phototherapy session, wherein the dosage is equivalent to a product of the intensity of the radiation source and an exposure time of the radiation source, wherein the dosage has an upper bound less than 1 minimal erythema dose (MED), and wherein delivery of the dosage provides an immune response to treat the autoimmune disorder.

2. The phototherapeutic system of claim 1 wherein the radiation source is configured to filter out a substantial portion of UV energy outside of the target wavelength range.

3. The phototherapeutic system of claim 1 wherein the radiation source is configured to expose at least 30% of a patient's skin to the light emitted by the radiation source.

4. The phototherapeutic system of claim 1 wherein the radiation source is a low-energy radiation source and is configured to be positioned within 3 cm of a treatment area.

5. The phototherapeutic system of claim 4 wherein the radiation source comprises an array of LEDs.

6. The phototherapeutic device of claim 1, further comprising:
a wearable substrate, and
wherein the radiation source comprises a plurality of LEDs arranged on the wearable substrate and configured to emit light within a treatment area.

7. The phototherapeutic device of claim 6 wherein the LEDs are configured to emit a substantially uniform UV radiation across the treatment area.

8. The phototherapeutic device of claim 6, further comprising a sensor on the wearable substrate, wherein the sensor is configured to determine proximity of the radiation sources to a patient's skin.

9. The phototherapeutic device of claim 1, further comprising a sensor configured to measure skin absorption, color, and/or reflection, wherein the controller is configured to select dosage based on the skin absorption, color, and/or reflection measured by the sensor.

10. The phototherapeutic device of claim 1 wherein the radiation source comprises a plurality of high-energy radiation sources configured to emit light of substantially equal intensity to the treatment area.

11. The phototherapeutic device of claim 10 wherein the plurality of high-energy radiation sources are configured to be spaced apart from the treatment area by about 10-200 cm, and wherein variations in distances between the high-energy radiation sources and the treatment area are less than 50 cm.

12. The phototherapeutic system of claim 1 wherein the radiation source comprises at least one of a narrow-band UVB source or a broad-band UVB source.

13. The phototherapeutic system of claim 1 wherein the dosage of the radiation source is configured to produce at least one of Adrenocorticotropic Hormone (ACTH), Melanocyte Stimulating Hormone (MSH), or Beta Endorphin (BE).

14. The phototherapeutic system of claim 1 wherein the dosage of the radiation source is configured to produce at least one of cis-urocanic acid or DNA pyrimidine dimers.

15. The phototherapeutic system of claim 1 wherein the intensity of the radiation source is an erythemally weighted irradiance equal to a summation of the product of an absolute measured intensity for each wavelength of light emitted by the radiation source and an erythema reference action spectrum weighting factor.

16. The phototherapeutic system of claim 1 wherein the radiation source comprises:
a UV radiation source and configured to emit energy;
a filter forward of the UV radiation source and configured to remove energy outside of the target wavelength range; and
a lens forward of the filter and configured to diffuse energy in a substantially uniform manner.

17. The phototherapeutic system of claim 1 wherein the radiation source comprises:
a UV radiation source; and
a lens forward of the UV radiation source, wherein the lens includes a filtering portion facing the UV radiation source and configured to remove light outside of the target wavelength range and lensing element spaced apart from the UV radiation source by the filtering portion and configured to diffuse filtered light in a substantially uniform manner.

18. A phototherapeutic system for treating an autoimmune disorders, the phototherapeutic system comprising:
a radiation source configured to emit light and having an intensity, wherein at least 75% of the light emitted by the radiation source has a target wavelength range with a bandwidth between 298 nm and 307 nm; and
a controller operably connected to the radiation source and configured to determine a dosage for a phototherapy session, wherein the dosage is equivalent to a product of the intensity of the radiation source and an exposure time of the radiation source, wherein the dosage has an upper bound less than 10 standard erythema dose (SED), and wherein delivery of the dosage provides an immune response to treat the autoimmune disorder.

19. A method of treating autoimmune disorders with a phototherapy system, the method comprising:
determining a skin type of a user;
determining, via a controller, a dosage of phototherapy to deliver to the user during a phototherapy session, wherein the dosage is equivalent to a product of the intensity of a radiation source of the phototherapy device and an exposure time of the radiation source, and wherein the dosage has an upper bound less than 1 minimal erythema dose (MED); and
delivering the dose of phototherapy to a treatment area on the user via the phototherapy device, wherein delivering the dose of phototherapy comprises emitting light from the radiation source having one or more target wavelength ranges within a bandwidth of 298-307 nm, wherein delivery of the dose of phototherapy provides an immune response to treat the autoimmune disorder.

20. The method of claim 19 wherein delivering the dose of phototherapy produces at least one of Adrenocorticotropic Hormone (ACTH), Melanocyte Stimulating Hormone (MSH), or Beta Endorphin (BE).

21. The method of claim 19 wherein delivering the dose of phototherapy produces at least one of cis-urocanic acid or DNA pyrimidine dimers.

22. The method of claim 19 wherein determining the skin type of the user comprises measuring, via a sensor, skin reflectance, color, or absorption of the user.

23. The method of claim 19, further comprising determining the intensity of the radiation source by summing the product of an absolute measured intensity for each wavelength of light emitted by the radiation source and an erythema reference action spectrum weighting factor.

24. The method of claim 19 wherein:
delivering the dose of phototherapy comprises emitting light from a plurality of high-energy radiation sources; and
the method further comprises positioning the treatment area of the user apart from the radiation sources by less than 200 cm, wherein variations in distance between the high-energy radiation sources and the treatment area are less than 50 cm.

25. The method of claim 19 wherein delivering the dose of phototherapy comprises delivering the dose of phototherapy to at least 30% of the user's skin.

26. The method of claim 19 wherein:
delivering the dose of phototherapy comprises emitting light from a plurality of low-energy radiation sources arranged on a wearable substrate; and
the method further comprises positioning the treatment area of the user apart from the low-intensity radiation sources by less than 3 cm and maintaining a substantially uniform distance between the treatment area and the radiation sources during the exposure time.

27. The method of claim 19, further comprising adjusting, via the controller, exposure time and intensity of the radiation source in relation to each other to select the dosage.

28. The method of claim 19, further comprising filtering out a substantial portion of UV energy outside of the target wavelength range.

29. The method of claim 19 wherein determining dosage of phototherapy comprises delivering the dosage of phototherapy based on the skin type of the user.

30. The method of claim 19, further comprising:
storing the skin type of the user on a database remote from the phototherapy device; and
accessing the skin type of the user during subsequent phototherapy sessions to determine the dosage of phototherapy.

31. The method of claim 19 wherein delivering the dose of phototherapy comprises:
filtering the light emitted from the radiation source to remove light outside of the target wavelength ranges; and
diffusing the filtered light with a lens to distribute the filtered light in a substantially uniform manner.
